(19) United States
(12) Patent Application Publication
(10) Pub. No.: US 2009/0298151 A1
(43) Pub. Date: Dec. 3, 2009

(54) HYDROGEN PRODUCING MICROORGANISM USEFUL FOR ENERGY GENERATION FROM DIVERSE CARBONACEOUS FEEDSTOCK

(75) Inventors: Elmar Schmid, Escondido, CA (US); James Gibson, Escondido, CA (US)

Correspondence Address: Patentique PLLC, P.O. Box 5803, Bellevue, WA, 98006, US

(73) Assignee: Dr. Elmar Schmid and James Gibson, Escondido, CA (US)

(21) Appl. No.: 12/132,574

(22) Filed: Jun. 3, 2008

(51) Int. Cl. C12P 3/00; C12N 1/20; C12N 15/74; C12N 15/01

(52) U.S. Cl. 435/168; 435/252.3; 435/471; 435/472; 435/446; 435/448; 435/443; 435/473; 435/441

(57) ABSTRACT

The disclosed invention relates to an isolated hydrogen gas producing microorganism, termed Enterobacter sp. SGT-T4™ and derivatives thereof. Compositions and methods comprising the disclosed microorganisms are also provided.

### FIELD OF THE INVENTION

The disclosed invention relates to the field of hydrogen gas production utilizing suitable microorganisms. This is an environmentally friendly and sustainable form of hydrogen-based energy production that is just starting to benefit human society. The disclosure describes an isolated and genetically unique microorganism, termed Enterobacter sp. SGT-T4 ™ The microorganism is metabolically versatile and generates high amounts of hydrogen gas from different renewable feedstock, including cellulosics- and hemicellulosics-derived sugars, alcoholic sugars, glycerol and glycerol-containing wastes as carbonaceous feedstock. The high hydrogen production rate of the disclosed microorganism with different feedstock is further increased in the presence of metallosilicates, such as natural zeolite. The disclosed microorganism implemented into a suitable bio-reactor environment will allow economical generation and on-site utilization of bio-hydrogen energy. This bio-hydrogen will be converted to electricity and heat by suitable means, such as a fuel cell. Sites with traditionally high amounts of cellulosics, hemicellulosics, starch, glycerol and other renewable bio-waste materials will have the ability to produce large amounts of bio-hydrogen.

### BACKGROUND OF THE INVENTION

Due to the eminent danger of global warming caused by rising anthropogenic discharge of fossil fuel-derived green house gases, most prominently carbon dioxide (CO₂), into earth's atmosphere and due to escalating costs for non-renewable fossil fuels, most namely petroleum and natural gas, there is an urgent need to find ecologically more friendly fuels. Biofuels, such as bio-ethanol from corn (maize) or sugarcane and bio-diesel produced from diverse plant oils, e.g. rapeseed or palm oil, have been increasingly heralded as attractive sustainable alternatives to the currently used fossil fuels, such as petroleum, coal and natural gas. Large investments have been put into bio-ethanol and bio-diesel plants in the past years. Another alternative and attractive fuel candidate with much less media attention is hydrogen gas (H₂). Hydrogen is the single most abundant chemical element in the universe and huge amounts of hydrogen atoms are conserved in the chemical bonds of renewable biomass, such as green plants-derived sucrose, cellulose, hemicellulose, starch, lipids and fats. Combustion of hydrogen gas results in the formation of water with no emission of the green house gas carbon dioxide (CO₂) as opposed to burning fossil fuels, bio-diesel and bio-ethanol. Moreover, hydrogen gas can be directly converted to electricity with high conversion efficiencies using fuel cell technologies. Generation of hydrogen gas from suitable high hydrogenated materials can be achieved by different means, including electrochemical, steam reforming, or with biological organisms. Electrochemical generation of hydrogen gas from water requires high energy inputs to achieve the necessary hydrolysis. Industrial scale hydrogen gas production from fossil fuels by steam reforming or coal gasification bears the disadvantage that this process is accompanied with high emissions of the green house gases (GHG) carbon dioxide (CO₂) and nitrogen oxides (NOₓ) as well as of the highly poisonous carbon monoxide (CO). Therefore there is a high interest in developing economical hydrogen gas-generating technologies which are ecologically more advantageous. These technologies also show real benefits for the mandated global carbon dioxide abatement. Hydrogen energy concepts and technologies have to be developed which allow cost-effective sequestration of CO₂, and which allow hydrogen gas generation from renewable resources, e.g. plant biomass, to assure a closed carbon cycle.

An interesting and currently overlooked alternative method of hydrogen generation is with the use of microorganisms. Bio-hydrogen production has been reported for a series of morphologically and genetically different microorganisms, including photosynthetic organisms, e.g. Rhodobacter sp. bacteria and the single-celled green algae Chlamydomonas reinhardtii, cyanobacteria, e.g. Oscillatoria sp. and several heterotrophic bacterial genera. The use of microorganisms for large scale production of bio-hydrogen gas has many advantages over the currently favored industrial scale generation of hydrogen gas from fossil fuels, e.g. gasification of coal. Most notably it is an environmentally clean method. Microbial hydrogen production can be sustainable with renewable biomass and/or derivatives thereof and can be conducted at ambient temperatures and pressures under comparatively low cost conditions (Hallenbeck, P. C. Water Sci Technol. 52(1-2):21-29 (2005); Nandi, R. et al., Critical Reviews in Microbiology 24(1):61-84 (1989)). Moreover, microbial hydrogen production is—with the exception of some thermophilic bacteria—not accompanied with the release of toxic and/or noxious gases, such as carbon monoxide (CO) and hydrogen sulfide (H₂S).

Several small scale operating bio-hydrogen generating platforms have been established and studied in the past decades utilizing different microorganisms and using various renewable materials from municipal solid wastes (MSW), food and packaging wastes, paper sludge hydrolysate, agriculture and forestry wastes, these microorganisms including Clostridia sp., Enterobacteria sp., Thermotogae sp., (Hallenbeck, P. C. Water Sci Technol. 52(1-2):21-29 (2005); Nandi, R. et al., Critical Reviews in Microbiology 24(1):61-84 (1989); Roychowdhury, S. et al., Int. J. Hydrogen Energy 13:407ff (1988)). However, to date none of the studied hydrogen-generating microorganisms and methods have lead to the successful introduction of an industrial scale bio-hydrogen production system. There are disadvantages and currently unsolved challenges with the mentioned microbes. For example, hydrogen gas generation with the help of photosynthetic microorganisms requires rather expensive incubation vessels with large, light-exposed surface areas and large quantities of increasingly expensive water. Effective hydrogen production in photosynthetic microorganisms is further hampered by low hydrogen production rates due to concomitantly released oxygen gas during the photosynthesis process. Heterotrophic bacteria have the advantage that they do not need solar energy and elaborated fermentation vessels for hydrogen production, but they are dependent on a suitably cheap, usually carbonaceous feedstock to assure low cost hydrogen production.

However, a significant disadvantage of heterotrophs is that the feedstock has to be supplied continuously and under contamination-free conditions to assure long term generation of hydrogen gas in the comparatively low cost fermentation vessels. Despite the fact that high and continuous hydrogen production rates have been shown for a series of heterotrophic microorganisms, including Klebsiella oxytoca (Minnan L. et al., Res. Microbiol. 156(1):76-81 (2005)), Thermotoga neapolitana (Van Ooteghem S. A. et al., Appl. Biochem. Biotechnol. 98-100:177-89 (2002)). Thermotoga elfii (Van Niel, E. W. J. et al., Int. J. Hydrogen Energy 27:1391-1398 (2002)), Caldicellulosiruptor saccharolyticus (Kadar Z. et al., Appl. Biochem. Biotechnol. 113-116:497-508 (2004)), Clostridia sp.(Ogino H. et al., Biotechnol. Prog. 21(6): 1786-1788 (2005), Enterobacter cloacae (Kumar N. et al., Process Biochemistry 35: 589-593 (2000) and Enterobacter aerogenes (Ito T. et al., J. Biosci. Bioeng. 97(4): 227-232 (2004), Ogino H. et al., Biotechnol. Prog. 21(6): 1786-1788 (2005), Taguchi, F. et al., U.S. Pat. No. 5,350,692 (Sep. 27, 1994)), under experimental lab conditions and with mostly purified glucose as the feedstock, no long term generation of hydrogen gas has been reported for any of the known hydrogen producers with cheap waste feedstock to date. Continuous high hydrogen production by known strictly anaerobic hydrogen producing bacteria, such as Clostridia sp. and Thermotoga sp., is hampered by the introduction of oxygen gas, a growth toxin to these microorganisms, usually carried in with the continuously supplied feedstock. Another major obstacle which prevented the successful industrial scale use of heterotrophic microorganisms for cost-effective generation of hydrogen gas from cheap waste feedstock is the high risk of contamination of the reaction vessel from the continuously supplied feedstock material.

Therefore, a facultative anaerobic and robust microorganism with high tolerance for oxygen levels and high hydrogen production from cheap feedstock would be advantageous for an industrial-scale biohydrogen production system. Furthermore, even though a series of mesophilic and moderate thermophilic microorganisms have been studied intensively for quantitative bio hydrogen production from common feedstock such as glucose, sucrose and maltose, no reports exist for more versatile bacteria capable of generating high amounts of hydrogen gas from other renewable biomass-derived feedstock, such as sucrose, maltose, xylose, arabinose, galactose, mannitol, sorbitol and glycerol.

Plant-derived cellulose and hemicellulose-containing materials (often referred to as cellulosics and hemicellulosics respectively) are the single most abundant renewable carbon source on earth and are annually produced by photosynthetic organism, such as grasses, shrubs and trees, on a Giga ton scale. Globally green plants convert about 190 Giga tons of carbon dioxide annually into renewable biomass mostly in the form of leaves, stems, wood, tubers and fruits. Industry-processed cellulosics, such as paper, newsprints, card board, and shopping bags, make up more than 40% of all municipal solid waste, a waste stream that to the vast extent ends up in land fills. Moreover, plant-derived oils serve as raw materials for the rapidly growing bio-diesel fuel industry which uses these renewable molecules to synthesize its biofuel using chemical methods. 3.8 million tons of bio-diesel was produced in 2005 via transesterification of oils that were extracted from a huge variety of sources including canola (rapeseed), corn, palm oil, and olives. Since glycerol is—together with salts and methanol—one of the major waste products generated during transesterification, it has in recent years flooded the glycerol market in the form of bio-diesel waste, lowered the glycerol price and started to generate a “glycerol waste problem”. In this respect it is of interest to know that for every tonne (=metric ton) of bio-diesel manufactured via the transesterification process, about 100 kg of glycerol waste is generated. Even though glycerol has traditionally been used in pharmaceuticals, cosmetics, toothpaste, paints and other commercial products, the rapidly developing bio-diesel industry with its large glycerol waste streams created a challenge to find profitably novel uses for this waste. Therefore, metabolically versatile microorganisms capable of generating hydrogen gas from glycerol and glycerol waste streams, such as bio-diesel wastes, could make significant contributions to generate clean bio-hydrogen energy.

A significant obstacle that hindered the successful and competitive use of microorganisms for industrial scale bio-hydrogen generation is the low hydrogen production rates and yields of the currently favored bacteria. To the knowledge of the authors of this document, no studies exist that show the rate- and yield-increasing effect of silicates, most prominently zeolites, on bacterial hydrogen production, even though many different strategies have been tried in the past to significantly increase the low hydrogen production rates and yield of bacteria. These include process optimization and genetic engineering. Zeolites are quite common crystalline aluminosilicate minerals with more than forty natural zeolites known today. Clinoptilolite, a naturally occurring zeolite and the most researched of all natural zeolites, has a cage-like structure consisting of SiO₄ and AlO₄ tetrahedra which are joined by shared oxygen atoms. Since the negative charges of the AlO₄ units of zeolites are balanced by the presence of exchangeable cations, usually sodium, potassium, calcium, magnesium, and iron, which can be easily replaced by other ions, zeolites possess high cation exchange and ion absorptive capacity. Despite their diverse known roles as filter material, absorbants and chemical catalysts, this invention shows a novel function of zeolites as cheap, abundant and very effective bacterial hydrogen production rate and yield increasing material.

The citation of documents herein is not to be construed as reflecting an admission that any is relevant prior art. Moreover, their citation is not an indication of a search for relevant disclosures. All statements regarding the date(s) or contents of the documents is based on available information and is not an admission as to their accuracy or correctness.

### BRIEF SUMMARY OF THE INVENTION

The disclosure is based on the isolation and characterization of a microorganism, referred to as Enterobacter sp. SGT-T4™ herein. The microorganism produces high amounts of hydrogen gas (or molecular hydrogen, H₂) from diverse carbon-made (or carbonaceous) feedstock and belongs to the bacterial family of enterobacteriaceae, a very ubiquitous and versatile group of gram-negative, facultative anaerobic bacteria. Enterobacteria are known to be metabolically versatile and are able to gain cell energy via respiratory (aerobic) or fermentative (anaerobic) degradation of a wide variety of different carbon containing molecules as starting materials. Enterobacteria which commonly occur in soil, water, sewage, food and are also found as normal intestinal inhabitants of humans and animals, are well studied and known to catabolize D-glucose and other carbohydrates, including L-arabinose, cellobiose, maltose, D-xylose, L-rhamnose, D-mannitol, D-sorbitol and trehalose. They are also known to produce organic acids and gas. Some enterobacterial species are known to generate hydrogen gas from other carbon-made molecules, such as pyruvate and glycerol. Glucose can be derived from many sources, but it is very abundant in green plants and in other renewable biomass-derived materials where it usually appears in the form of the disaccharide sucrose and of the polysaccharides starch and cellulose. Other monosugars, most prominently arabinose, xylose, galactose and rhamnose are common components of the hemicellulose and pectin fraction of renewable biomass, e.g., green plants and other phototrophic organisms. Another important renewable biomass-derived component is the 3-carbon molecule glycerol which is an integral compound of plant- or animal-derived oils, lipids and fats.

In one aspect, the disclosure includes a hydrogen producing microorganism as described herein. Non-limiting examples of microorganisms of the disclosure includes a microorganism comprising a 16S rDNA sequence fragment represented by SEQ ID No:1 (Table 5). The disclosure thus includes a microorganism of the enterobacteriaceae family which generates high amounts of hydrogen gas from carbohydrates derived from a diverse range of starch, cellulose, and hemicellulose containing materials, or a combination of two or more of such materials. In some embodiments, a disclosed microorganism of the enterobacteriaceae family utilizes one or more of the carbon containing compounds listed above. In some cases, the microorganism generates large amounts of hydrogen gas and at a high rate from glycerol and glycerol-containing feedstock, for example bio-diesel waste.

In a second aspect, the disclosure includes a method of culturing a microorganism as described herein. In some embodiments, the microorganism is cultured with one, two or more carbon containing compound, including one or more carbohydrates as a non-limiting example, under defined cultivation conditions. In other embodiments, the disclosed microorganism is cultured under conditions that allow high production rates of hydrogen gas, such as by use of the carbohydrate(s). The disclosure thus includes a method of producing hydrogen gas by cultivating a disclosed microorganism. In further embodiments, hydrogen gas production is based upon growth of a disclosed microorganism on the glycerol content of waste streams derived from bio-diesel production. In some cases, the glycerol is produced by transesterification or other methods known in the field of producing the bio fuel.

The disclosure includes additional embodiments of a method of culturing a microorganism. In some embodiments, a disclosed microorganism is cultured with alcoholic sugars as feedstock, e.g. mannitol, under defined cultivation conditions. In other embodiments, a disclosed microorganism is cultured under conditions that allow high production rates of hydrogen gas, such as by use of the alcoholic sugars. In further embodiments, hydrogen gas production is based upon extracted alcoholic sugar products of brown algae (kelp) extracts.

In other embodiments, the disclosure includes a method of culturing a microorganism as described herein with the tertiary alcohol glycerol as feedstock under defined cultivation conditions. In some embodiments, a disclosed microorganism is cultured under conditions that allow high production rates of hydrogen gas, such as by use of glycerol. In further embodiments, hydrogen gas production is based upon cultivation of the microorganism in the presence of crude, extracted bio-diesel production wastes containing glycerol.

In most embodiments, a cultivation condition used in a disclosed method includes the use of an aqueous based culture medium, or aqueous environment. In some embodiments, a cultivation condition includes the presence of inorganic salts. In some cases, the salts are in milligram or microgram amounts, such as by addition of exogenous salts to a culture medium. Non-limiting examples of the salts include those containing iron, selenium, molybdenum, nickel, magnesium, zinc, manganese, copper, borate and/or cobalt. In other embodiments, a cultivation condition includes the presence of known co-substrates or prosthetic groups of crucial metabolic enzymes and other bio-catalysts. Non-limiting examples include nicotinic acid, nicotine amide, riboflavin, biotin, and/or thiamin, which may be exogenously added to a culture medium for use in a disclosed method. In yet other embodiments, a cultivation condition includes the presence of sulfur-containing compounds. Non-limiting examples include ammonium sulfate, cysteine, methionine, glutathione, N-acetyl cysteine and/or dithiothreitol, which may be exogenously added to a culture medium for use in a disclosed method.

In further embodiments, a cultivation condition includes redox-active compounds and/or compounds with antioxidant chemical characteristics. In some cases, the amount of such a compound is defined in the culture medium. Non-limiting examples of such a compound include ascorbic acid, tocopherols, cysteine, N-acetyl cysteine and/or glutathione.

In yet additional embodiments, a cultivation condition includes the presence of highly absorptive materials, crystals, minerals and/or mineral-like compounds. In some cases, the material is a metallosilicate, such as an aluminosilicate, and the amount of such a material or mineral is optionally defined in the culture medium. In other cases, such a material or mineral is added to the culture medium in granular, microgranular and/or nanogranular form. Non-limiting examples of a highly absorptive material or mineral include cellulose fibers, diatomaceous earth, Celite®, natural zeolite (clinoptilolite), a synthetic zeolite, silicon dioxide, titanium dioxide, zirconium dioxide, and/or cerium dioxide.

A cultivation condition of the disclosure may also include the presence of a gaseous phase above the culture medium. The gas phase may be optionally continuously flushed, or replenished, with a desired gas. In some embodiments, the desired gas does not contain oxygen. In other embodiments, the desired gas is a noble gas, such as argon as a non-limiting example. In alternative embodiments, the gas is flushed in a discontinuous manner, such as at defined times, during the culturing of the microorganism with the desired gas. In further embodiments, the desired gas is bubbled through the aqueous environment, or culture medium. The bubbling may be continuous or discontinuous, such as at defined time points during the culturing of the microorganism.

In some embodiments, the introduction of gas may be used to remove carbon dioxide generated by the cultivation conditions. Alternatively, carbon dioxide may be chemically bound to an absorbent present under the cultivation conditions. In some cases, the absorbent is an alkali metal liquid matrix. Non-limiting examples include sodium hydroxide (NaOH), and/or a solid matrix, such as soda lime.

A cultivation condition of the disclosure also includes a temperature, salinity and a pH level (each of which is optionally defined), suitable for the growth and/or propagation of the microorganism as well as hydrogen gas production. In some embodiments, the temperature is maintained at or below about 45° C. In other embodiments, the salinity of the medium is maintained at a concentration of less than 6%. In other embodiments, the pH is maintained at a level from about 4.5 to about 7.5, such as at about 5.0, about 5.5, about 6.0, about 6.5, or about 7.0.

A cultivation condition of the disclosure may also include the continuous supplying of a liquid feedstock, or medium, to the microorganism. In some embodiments, the feedstock contains at least one component selected from monosaccharides, disaccharides, polysaccharides, alcoholic sugars, amino acids, glycerol, fatty acids, and combinations thereof. Non-limiting examples of monosaccharides and disaccharides include glucose, sucrose, maltose, cellobiose, other saccharides containing glucose units, or any combination of the foregoing. In some embodiments, a feedstock contains arabinose, xylose, galactose, rhamnose, sorbitol, mannitol or any combination of the foregoing. In other embodiments, a feedstock contains glycerol, monoacylglycerides, diacylglycerides or any combination of the foregoing.

Therefore, an additional aspect of the disclosure is a culture medium or formulation for use in a method as described herein. The medium or formulation may be a complex or enriched, or alternatively defined or synthetic, growth media which supports hydrogen gas production by a disclosed microorganism. In some embodiments, the medium or formulation allows maximum, as compared to other media or formulations, hydrogen gas production under the conditions used. In other embodiments, the medium or formulation is the defined or synthetic which allows for maximum hydrogen gas production. In some embodiments, the medium or formulation contains defined amounts of absorptive materials or minerals.

In a further aspect, the disclosure includes a method of producing energy. The method may comprise producing hydrogen gas with a disclosed microorganism and supplying the hydrogen gas to a hydrogen gas energy converting device. Non-limiting examples include a fuel cell, gas turbine, internal combustion engine or other suitable hydrogen energy conversion device. The converting device may convert the hydrogen gas to either kinetic energy or potential energy. Kinetic energy is based on motion including that of waves, electrons, atoms, molecules, substances, and objects. Non-limiting examples of kinetic energy include electrical energy, radiant energy, thermal energy, motion energy, and sound. Potential energy is stored energy and the energy of position. Non-limiting examples of potential energy include chemical energy, stored mechanical energy, nuclear energy, and gravitational energy.

In a yet further aspect, the disclosure includes a method of identifying, or detecting a disclosed microorganism. In some embodiments, the method comprises identifying or detecting a microorganism as comprising a 16S rDNA sequence containing a sequence with more than 87% homology to SEQ ID No:1 (Table 5). Non-limiting examples include identifying or detecting a microorganism as comprising a 16S rDNA containing SEQ ID No:1.

In other embodiments, the method comprises identifying or detecting a microorganism as containing a sequence which is amplified by a pair of primers comprising sequences represented by SEQ ID No: 2 and SEQ ID No: 3 (Table 4). The method may comprise use of the two sequences as the primers in a polymerase chain reaction (PCR) with DNA from a candidate microorganism followed by comparison of the amplified sequence with that amplified from SGT-T4™. Non-limiting examples include comparison of the length or base composition of the amplified nucleic acid, or of the sequence of amplified nucleic acid. Optionally, the method may further comprise assaying the candidate microorganism for hydrogen gas production.

The method of identifying or detecting may be of a candidate microorganism isolated from a naturally occurring source or as it is found in nature. Alternatively, the method may be performed with a candidate microorganism derived from a microorganism disclosed herein. In some embodiments, such a derivative, or mutant, microorganism may be one which occurs with passage of a disclosed microorganism in culture. Alternatively, a derivative microorganism may be the result of intentional mutagenesis of a disclosed microorganism.

In other embodiments, the disclosure includes a method of mutagenizing, or creating, derivative microorganisms from a disclosed microorganism. The method may comprise taking a disclosed microorganism and contacting it with a mutagen. Non-limiting examples of mutagens include mutagenic agents, such as chemical compounds, and radiation. The method may further comprise screening the treated microorganism(s) for an rDNA sequence as described herein and/or production of hydrogen gas. In some embodiments, the screening may comprise detection of increased hydrogen gas production. Non-limiting examples of increased production include an increased rate of production over a given period of time and/or increased total gas production over a given period.

Another aspect of the disclosure includes nucleic acid molecules for use in the methods as described herein. In some embodiments the molecules are isolated from the cellular or genomic DNA environment in which they are normally found. A non-limiting molecule is represented by SEQ ID No: 1 (Table 5). In other embodiments, the molecule may be a vector or plasmid, such as one comprising the molecules represented by SEQ ID No: 1. Other molecules of the disclosure are represented by SEQ ID Nos: 2 and 3 (Table 4).

The details of additional embodiments are set forth in the accompanying drawings and the description below. Other features, objects, and advantages of the embodiments will be apparent from the drawings and detailed description, and from the claims.

### BRIEF DESCRIPTION OF THE DRAWINGS

FIG. 1a shows the comparative time-dependent total gas production of the bacterium Enterobacter sp. SGT-T4™ in comparison to the known gas producing enterobacteria Enterobacter sp. SGT06-1™ and Enterobacter aerogenes ATCC 13048. In this study, the bacteria under investigation were incubated at 37° C. in test tubes containing inverted Durham tubes filled with complex growth medium (10 ml) containing peptone and 2.5% glucose as feedstock. Gas production of the bacteria was measured and plotted as mm trapped gas in the inverted Durham tubes over time.

FIG. 1b shows the comparative time-dependent total gas production of the bacterium Enterobacter sp. SGT-T4™ in different growth media in the presence or absence of natural zeolite. In this study, SGT-T4™ bacteria were incubated at 37° C. in test tubes containing inverted Durham tubes filled with 10 ml of either peptone-glucose (PG) medium (=Medium 1) or with 10 ml tryptone-yeast-glucose (TYG) medium (=Medium 2). Gas production of the bacteria over time was measured and plotted as mm trapped gas in the inverted Durham tubes. The concentration of glucose and zeolite (Zeo) in the media was 2.5% in this study.

FIG. 2a shows the time-dependent hydrogen gas generation of the bacterium Enterobacter sp. SGT-T4™ in TYG medium in the presence or absence of natural zeolite. In this study, SGT-T4™ bacteria were incubated at 37° C. under microaerobic conditions in rubber-stoppered flasks filled with 50 ml of tryptone-yeast-glucose (TYG) medium. Gas production of SGT-T4™ over time was measured with the help of a liquid-gas exchange method consisting of an upside-down graduated cylinder filled with a 15% NaOH solution that trapped the carbon dioxide fraction of the evolved gas. In this study the concentration of glucose and zeolite (Zeo) in the media was 2% and 2.5%, respectively.

FIG. 2b shows the time-dependent hydrogen gas production rate of the bacterium Enterobacter sp. SGT-T4 ™ calculated in ml H₂ evolved per hour per liter growth medium. SGT-T4 ™ was incubated under the same conditions as described in FIG. 2a. in TYG medium in the presence or absence of natural zeolite.

FIG. 3a shows the time-dependent total gas production of Enterobacter sp. SGT-T4™ in the presence of the monosaccharides glucose, xylose, arabinose, galactose or of the disaccharides maltose and sucrose as feedstock. In this study, SGT-T4™ was incubated at 37° C. in test tubes containing inverted Durham tubes filled with peptone growth medium (10 ml) with 2.5% of the carbohydrates as feedstock. Gas production was measured and plotted as mm trapped gas in the inverted Durham tubes over time.

FIG. 3b shows the time-dependent total gas production of Enterobacter sp. SGT-T4™ in the presence of the alcoholic sugars mannitol or sorbitol or of the polyhydroxyalcohol glycerol as feedstock. In this study, SGT-T4™ was incubated at 37° C. in test tubes containing inverted Durham tubes filled with peptone growth medium (10 ml) with 2.5% of the feedstock. Gas production was measured and plotted as mm trapped gas in the inverted Durham tubes over time.

FIG. 4 shows the time-dependent total gas production of Enterobacter sp. SGT-T4™ with industrial glycerol or crude bio-diesel waste (BDW) as feedstock in the presence or absence of zeolite (Zeo) in the growth medium. In this study, SGT-T4™ was incubated at 37° C. in tryptone-yeast (TY) medium (10 ml) in the presence of either 300 mM glycerol or 0.8 ml of crude bio-diesel waste (BDW) as carbon feedstock. Gas production was measured in the presence or absence of 2.5% of zeolite material in the growth medium and plotted as mm trapped gas in the inverted Durham tubes over time. The known high gas production of Enterobacter sp. SGT-T4™ with glucose as feedstock is shown for comparison.

FIG. 5a shows the time-dependent hydrogen gas (H₂) production of Enterobacter sp. SGT-T4™ with 300 mM industrial glycerol or 3.8 ml of crude bio-diesel waste (BDW) as carbon feedstock in the presence or absence of 2.5% zeolite (Zeo) in the growth medium. In this study, SGT-T4 ™ bacteria were incubated at 37° C. under microaerobic conditions in rubber-stoppered flasks filled with 50 ml of tryptone-yeast (TY) medium. Gas production of SGT-T4 ™ over time was measured with the help of a liquid-gas exchange method consisting of an upside-down graduated cylinder filled with a 15% NaOH solution that trapped the carbon dioxide fraction of the evolved gas. The time-dependent H₂ production of Enterobacter sp. SGT-T4™ with 2.5% glucose as feedstock is shown for comparison.

FIG. 5b shows the time-dependent hydrogen production rates (in ml H₂ per hour per liter) of Enterobacter sp. SGT-T4™ with 300 mM industrial glycerol or with 3.8 ml of crude bio-diesel waste (BDW) as carbon feedstock in the presence or absence of 2.5% zeolite (Zeo) in the growth medium. The SGT-T4 ™ bacteria were incubated at 37° C. under the same conditions as described in more detail in FIG. 5a. The hydrogen production rate of glucose (2.5%) as feedstock in the absence of zeolite is shown for comparison.

FIG. 6a shows the increased gas production of Enterobacter sp. SGT-T4™ with pre-processed bio-diesel waste solution (BDWS) in comparison with crude bio-diesel waste (BDW) as feedstock in the presence or absence of zeolite (Zeo) in the growth medium. In the case of BDWS, SGT-T4™ was incubated at 37° C. in 5 ml 2×tryptone-yeast (TY) medium to which 5 ml of pre-processed bio-diesel waste solution (BDWS) as carbon feedstock was added. Pre-processed bio-diesel waste solution (BDWS) was prepared by dissolving 40 ml of pre-cleared bio-diesel waste (BDW) in 460 ml deionized water followed by pH-neutralization with 6NHC1. In the case of BDW as feedstock, 0.8 ml of crude bio-diesel waste (BDW) was directly added to 9.2 ml tryptone-yeast (TY) medium and the time-dependent accumulation of gas in the Durham tubes measured. Gas production was measured in the presence or absence of 2.5% of zeolite (Zeo) material in the growth medium and plotted as mm trapped gas in the inverted Durham tubes over time. The known high gas production of Enterobacter sp. SGT-T4™ with glucose as feedstock in the presence of 2.5% zeolite is shown for comparison.

FIG. 6b shows the effect of increasing concentration of zeolite (Zeo) material in the growth medium on the gas production of Enterobacter sp. SGT-T4™ with pre-processed bio-diesel waste solution (BDWS) as feedstock. In this study, SGT-T4™ was incubated at 37° C. in 5 ml 2×tryptone-yeast (TY) medium to which 5 ml of pre-processed bio-diesel waste solution (BDWS) was added as carbon feedstock. Pre-processed bio-diesel waste solution (BDWS) was prepared by dissolving 40 ml of pre-cleared bio-diesel waste (BDW) in 460 ml deionized water followed by pH-neutralization with 6NHC1. The accumulation of gas in the inverted Durham tubes in the absence or in the presence of defined amounts of zeolite (Zeo) material was measured after 7 hours incubation time and plotted as mm gas in the Durham tubes.

FIG. 7a shows the time-dependent hydrogen production rates (in ml H₂ per hour per liter) of Enterobacter sp. SGT-T4™ with pre-processed bio-diesel waste (BDWS) as feedstock in the presence or absence of 2.5% zeolite (Zeo). In this study, SGT-T4 ™ bacteria were incubated at 37° C. under microaerobic conditions in rubber-stoppered flasks filled with 25 ml of 2×-concentrated tryptone-yeast (TY) medium and 25 ml BDWS solution. Gas production of SGT-T4™ over time was measured with the help of a liquid-gas exchange method consisting of an upside-down graduated cylinder filled with a 15% NaOH solution that trapped the carbon dioxide fraction of the evolved gas. The BDWS solution was prepared by dissolving 40 ml of pre-cleared bio-diesel waste (BDW) in 460 ml of sterile distilled water followed by pH neutralization with HCl.

FIG. 7b shows the maximum hydrogen production rates, volumes and yield of Enterobacter sp. SGT-T4™ (in the presence or absence of 2.5% zeolite) in comparison to the published rates, volumes and yield of Enterobacter aerogenes HU-101 [Ito T., et al., J. Biosci. Bioeng. 100(3): 260-265 (2005)] and Klebsiella pneumonia DSM2026 [Liu F. & Fang B. Biotechnol. J. 2(3): 374-380 (2007)] with bio-diesel waste as feedstock. The numbers for Enterobacter sp. SGT-T4™ and Klebsiella pneumonia were from incubations of the microbes under batch conditions, while the shown numbers for Enterobacter aerogenes HU-101 result from continuous culturing of self-immobilized cells in a 60 ml packed-bed reactor (asterisk). The shown hydrogen gas volumes for the individual bacteria are given for a 50 ml fermentation volume and after 24 hours incubation at 37° C.

FIG. 8 shows the comparative sedimentation behavior of Enterobacter sp. SGT-T4™, Enterobacter sp. SGT06-1™ and Enterobacter aerogenes ATCC13048 (E.a.) in tryptone-yeast-glucose (TYG) medium. 15 hour cultures of the microorganisms were resuspended in the media by gentle shaking of the tubes and then left on the bench for 12 hours without further agitation of the tubes during this time period. The picture was taken after 12 hours.

### DETAILED DESCRIPTION OF THE INVENTION

### General

The disclosure is based in part on extensive investigations on an ideal source of hydrogen producing microorganisms and systematically screened guts dissected from different termite (white ant) species for the presence of metabolically versatile and high hydrogen producing microorganisms. The disclosure includes the successful isolation and characterization of a suitable candidate bacterium, termed Enterobacter sp. SGT-T4™. The isolated bacterium has favorably fast growth rates and generates very high amounts of hydrogen gas from glucose as carbon feedstock and also from other renewable biomass-derived carbonaceous molecules, such as glycerol, cellobiose, maltose, sucrose, arabinose, xylose, galactose, rhamnose and alcoholic sugars, such as mannitol and sorbitol. Stated differently, the disclosed microorganism is capable of generating hydrogen gas not only from starch and cellulosics-degradation products, such as glucose, maltose and cellobiose, or from hemicellulosics-derived monosugars, such as rhamnose, xylose, galactose and arabinose, but also from glycerol and alcoholic sugars. Without being bound by theory, and offered to improve the understanding of the disclosed embodiments, the microorganism generates hydrogen gas by fermentation of degradation products of cellulosics materials, such as paper and cotton waste streams, from hemicellulosics degradation products, such as green plant biomass, from alcoholic sugars, such as mannitol, the predominant storage sugar form in brown algae (phaeophytes) and from glycerol, a key component of biological lipids and fats and a major waste product of bio-diesel processing system. The already high hydrogen production rate of the disclosed bacterium with glucose, glycerol, and other carbonaceous feedstock can be further enhanced in the presence of highly absorptive and catalytically active materials, most prominently zeolite and diatomaceous earth, although another metallosilicate may be used.

One non-limiting example of a metallosilicate is a crystalline aluminosilicate, such as a zeolite. More than forty natural zeolites are known and contemplated for use in the practice of the disclosed methods and compositions. One non-limiting example is clinoptilolite, Also without being bound by theory, the isolated and characterized hydrogen gas-generating bacterium, termed SGT-T4™, is believed to belong to the genus Enterobacter. The bacterium, and derivatives thereof, as well as the cultivation conditions using absorptive materials as described herein, may be used for long term and large scale generation of hydrogen gas in combination with known or future energy conversion technologies, i.e. fuel cells and/or gas turbines.

The microorganisms and methods described herein contribute to the technical field of bio-energy generation from renewable biomass-derived molecules and components, i.e. sucrose, starch, glycerol, alcoholic sugars, as well as cellulose- and/or hemicellulose-containing materials.

Throughout this document, cellulose-containing materials are herewith referred to as cellulosics, e.g. paper waste, card board, cotton-made fabrics. Hemicellulose-containing materials, e.g. food processing wastes, agriculture and forestry plant biomass, will be termed hemicellulosics. The disclosed microorganism generates hydrogen gas in the presence of structurally diverse carbohydrates, including the monosaccharides glucose, mannose, xylose, arabinose, galactose, rhamnose, from the disaccharides cellobiose, maltose and sucrose, from the alcoholic sugar mannitol, from glycerol and also from glycerol-containing bio-diesel production wastes.

The disclosed bacteria and processes are suitable for utilization of sucrose, starch, cellulosics and hemicellulosics-derived carbohydrate feedstock, as well as waste streams rich in alcoholic sugars, e.g., mannitol, or glycerol, e.g. bio-diesel production wastes, for industrial scale bio-hydrogen gas production. Proposed industrial scale biohydrogen energy production systems will utilize the microorganism SGT-T4™, or a derivative thereof, at sites with traditionally large starch, cellulosics, hemicellulosics, and glycerol-containing waste loads, such as food processing industries, breweries, large office buildings, government offices, educational institutions, shopping malls, hospitals, farms, nurseries, and bio-diesel processing plants. The microorganism may also be utilized for industrial bio-hydrogen production from sources rich in alcoholic sugars, such as brown algae, and at sites with high amounts of alcoholic sugar-containing waste streams containing mannitol and/or sorbitol. The microorganism, cultivation methods and processes of the disclosure can be effectively used for on-site, decentralized industrial scale production of bio-energy in the form of electricity and/or heat from renewable materials under ultra-low green house gas-emitting conditions. Therefore, the disclosed invention is expected to make significant contributions to domestic energy security, air quality improvement, natural resource conservation, land use protection and pollution prevention.

Microorganisms

As described herein, the disclosure includes a microorganism belonging to the enterobacteriaceae family. The bacterium generates high amounts of hydrogen gas (H₂) from sucrose, different starch, cellulosics- and hemicellulosics-derived carbohydrates, namely glucose, maltose, cellobiose, xylose, rhamnose, galactose and arabinose, and glycerol in different culture media and under defined cultivation methods. In some embodiments, the hydrogen gas produced by the microorganism SGT-T4™ is directly used in a bio-reactor-coupled fuel cell system for long-term electricity generation under ambient temperatures. Because the microorganism only generates hydrogen and carbon dioxide gas from the supplied carbonaceous feedstock and does not release potentially noxious gases, such as hydrogen sulfide (H₂S) and carbon monoxide (CO). Both of these are known to cause fuel cell membrane poisoning. Because of these properties the microorganism SGT-T4™ is ideal for use in combination with fuel cell energy systems.

One non-limiting example of a disclosed microorganism is a hydrogen gas producing microorganism comprising a 16S rDNA sequence represented by SEQ ID No: 1. This microorganism is termed SGT-T4™, and it has been isolated from a naturally occurring source to be free of other microorganisms found with it in nature. The genetic material of the microorganism may be further isolated and sequenced by methods known to the skilled person to identify additional sequences that are unique, or specific, to the microorganism and/or hydrogen gas production. These additional sequences may also be used to identify or characterize additional hydrogen gas producing microorganisms of the disclosure.

Additional microorganisms of the disclosure include derivatives, or mutants, of SGT-T4™, such as those which occur spontaneously with passage or cultivation. In some cases, derivative microorganisms may be considered progeny microorganisms of SGT-T4™. In other cases, the derivative microorganisms are spontaneous mutants containing genetic changes at one or more locations in the genomes of SGT-T4™. Non-limiting examples of genetic changes includes insertion and/or deletion of sequences, and/or substitution of one or more base residues. In many embodiments, the derivative or mutant microorganisms retains the hydrogen gas production phenotype of SGT-T4™ and/or a 16S rDNA sequence as described herein.

Whether a derivative, a mutant, or isolated, a microorganism of the disclosure may be identified as comprising a 16S rDNA sequence containing SEQ ID No:1 (Table 5), or a sequence with more than 87% identity or homology to SEQ. ID No: 1. In other embodiments, the microorganism comprises a 16S rDNA sequence containing a sequence with more than 87%, more than 88%, more than 89%, more than 90%, more than 91%, more than 92%, more than 93%, more than 94%, more than 95%, more than 96%, more than 97%, more than 98%, or more than 99% identity to SEQ ID No:1. Of course some microorganisms may comprise SEQ ID No:1. Percent identity or homology between two sequences may be determined by any suitable method as known to the skilled person. In some embodiments, a PSI BLAST search, such as, but not limited to version 2.1.2 (Altschul, S. F., et al., Nucleic Acids Rec. 25:3389-3402, 1997) using default parameters may be used.

Hydrogen Gas Production and Use

In addition to culturing a disclosed microorganism with a suitable medium and conditions to propagate it, the disclosure also includes a method of culturing a microorganism as described herein to produce hydrogen gas. In some embodiments, the microorganism is cultured with a source of carbohydrate(s) as described herein. The method may also comprise cultivation conditions that are suitable or advantageous to hydrogen gas production, such as the use of a culture medium and/or conditions as described herein.

The disclosure thus includes a cell culture comprising a microorganism of the disclosure and a culture medium or formulation as described herein. In some embodiments, the medium or formulation includes the combination of a source of carbohydrate(s), one or more inorganic salts, a processed protein extract, yeast extract, a sulfur-containing compound, and a redox-active compound and/or antioxidant compound, each of which is as described herein. In further embodiments, a cell culture may contain defined amounts of one or a combination of absorptive materials, for example cellulose, cellulose-derivatives, natural zeolites (clinoptilolite), synthetic zeolites, diatomaceous earth, or other alumino- or metal silicates, and may be exposed to an absorbent for carbon dioxide as described herein. In further embodiments, a cell culture may contain defined amounts of one or a combination of solid catalytic materials, for example a natural zeolite (clinoptilolite), synthetic zeolites, or other alumino- or metal silicates, and the bio-reactor containing the catalytic materials may be exposed to a form of electromagnetic energy, for example to visible or UV light.

A cell culture may be maintained or propagated under conditions that include a combination of a gaseous phase above the medium or formulation, a suitable temperature, suitable agitation of the medium or formulation, suitable osmolarity, suitable salt concentration and an acceptable pH, each as described herein. In some cases, the gaseous phase comprises an inert or noble gas, which is optionally bubbled through a liquid medium or formulation. Non-limiting examples of a suitable temperature include at or below about 45° C. or about 40° C., about 37° C., about 35° C., about 30° C., or about 25° C.

The disclosure further includes a method of producing energy that comprises releasing energy from hydrogen gas produced by a disclosed method. In some embodiments, the method may comprise delivery of hydrogen gas produced by a disclosed microorganism and supplying the hydrogen gas to a hydrogen gas energy converting device. In some cases, the hydrogen gas releases energy during combustion in the presence of oxygen to form water. In other cases, the energy release occurs via electrochemical conversion, such as in a fuel cell with hydrogen gas as a fuel and oxygen as the oxidant.

The disclosure thus includes a method of producing molecular hydrogen (H₂) by culturing a disclosed microorganism under conditions allowing hydrogen production. In some embodiments, the conditions include an aqueous environment containing gram amounts of added alkali phosphates, yeast extract, malt extract, and/or a protein hydrolysate extract. Non-limiting examples of a protein hydrolysate extract include tryptone and peptone. In other embodiments, the conditions include an aqueous environment containing milli- or microgram amounts of added inorganic salts, such as calcium, magnesium, manganese, iron, selenium, molybdenum, nickel and/or zinc, or any combination thereof. In further embodiments, the conditions include an aqueous environment containing defined amounts of redox-active compounds and/or compounds with either antioxidant or oxidant chemical characteristics, such as ascorbic acid, N-acetyl cysteine, methionine, cysteine, glutathione, and/or hydrogen peroxide.

In additional embodiments, the conditions include a gas phase above an aqueous environment that is continuously flushed with gas, optionally of a defined amount or composition. In some cases, the gas is a noble gas, such as argon as a non-limiting example. Further embodiments include flushing over time, such as at defined time points, with gas (optionally of a defined amount or composition). Non-limiting examples include flushing with a noble gas like argon as a non-limiting example. Alternatively, the conditions may include an aqueous environment that is continuously bubbled with gas of a defined amount or composition, such as with a noble gas like argon as a non-limiting example. As a further alternatively, the conditions may include an aqueous environment that is flushed with gas of a defined amount or composition, such as a noble gas like argon as a non-limiting example.

In further embodiments, the conditions may include maintaining a culture environment at a constant or relatively constant temperature. In some cases, the temperature may be about 45° C. or below. In other cases, the temperature may be about 40° C. or below, about 37° C. or below, about 35° C. or below, about 30° C. or below, about 25° C. or below, or about room temperature or below.

Additional embodiments include conditions with a continuously supplied liquid feedstock. Non-limiting examples include feedstock derived from a monosaccharide, a disaccharide, a polysaccharide, an alcoholic sugar, a polyhydroxyalcohol, an amino acid, a fatty acid, and any combination thereof. In some cases, a mono- or di-saccharide is selected from glucose, sucrose, maltose, cellobiose and/or other saccharides containing a glucose unit or a combination thereof. In other cases, the feedstock contains arabinose, xylose, galactose, rhamnose, sorbitol and/or mannitol or any combinations thereof. In additional cases, the feedstock contains a polyhydroxyalcohol, such as glycerol, monoacylglycerol and/or diacylglycerol, or a combination thereof as non-limiting examples.

Embodiments of the disclosure further include conditions with generation of carbon dioxide in the culture environment. In some cases, the carbon dioxide is chemically bound or sequestered by inclusion of an alkali metal liquid, or solid, matrix in the environment. Non-limiting examples of a matrix include sodium hydroxide (NaOH) in solution and soda lime as a solid matrix.

Identification of Microorganisms

The disclosure includes a method of identifying, or detecting a disclosed microorganism based on the nucleic acid sequences of the microorganism, optionally in combination with the detection of hydrogen production by the microorganism. Thus in some embodiments, the method comprises identifying or detecting a candidate or test microorganism as comprising 16S rDNA containing a sequence with more than 87% identity or homology to SEQ. ID No:1, which identifies it as a microorganism of the disclosure. Microorganisms with such levels of sequence identity are described herein, and they include a microorganism comprising a 16S rDNA containing SEQ ID No:1.

In other embodiments, the method comprises identifying or detecting a microorganism as containing a 16S rDNA sequence which hybridizes to SEQ ID No:1 under “stringent conditions.” Hybridization refers to the interaction between two single-stranded nucleic acids to form a double-stranded duplex molecule. The region of double-strandedness may be full-length for both single stranded molecules, full-length for one of the two single stranded molecules, or not full-length for either of the single-stranded nucleic acids. “Stringent conditions” refer to hybridization conditions comprising, or equivalent to, 68° C. in a solution consisting of 5×SSPE, 1% SDS, 5×Denhardt's reagent and 100 μg/ml denatured salmon sperm DNA followed by washing in a solution comprising 0.1×SSPE, and 0.1% SDS at 68° C., or the above conditions with 50% formamide at 42° C. Stringent condition washes can include 0.1×SSC to 0.2×SSC, 1% SDS, 65° C., for about 15-20 min. A non-limiting example of stringent wash conditions is 0.2×SSC wash at 65° C. for about 15 minutes (see, Sambrook et al., Molecular Cloning—A Laboratory Manual (2nd ed.) Vol. 1-3, Cold Spring Harbor Laboratory, Cold Spring Harbor Press, NY, 1989, for a description of the SSC buffer). Other exemplary stringent conditions include 7% SDS, 0.25 M sodium phosphate buffer, pH 7.0-7.2, 0.25 M sodium chloride at 65° C. to 68° C. or such conditions with 50% formamide at 42° C.

The test or candidate microorganism may be isolated from a naturally occurring source or as found in nature. Alternatively, the method may be performed with a progeny microorganism derived from SGT-T4™. Alternatively, a derivative microorganism may be the result of intentional mutagenesis of a disclosed microorganism.

Mutagenesis Methods

The disclosure includes a method of mutagenizing, or creating, derivative microorganisms from a disclosed microorganism, such as SGT-T4™. The method may comprise taking one of the disclosed microorganisms and treating it with a mutagen. Non-limiting examples of a mutagen include UV or ionizing irradiation, a deaminating agent (such as nitrous acid), an alkylating agent (such as methyl-N-nitrosoguanidine (MNNG)), sodium azide, an intercalating agent (such as ethidium bromide), or phage, transposon or group II intron-mediated mutagenesis. In some embodiments, the method may further comprise the screening of the treated microorganism with a method described herein for the identification of microorganisms by detection of 16S rDNA sequences and/or recA sequences, optionally in combination with detection of hydrogen gas production.

In further embodiments, the mutagenesis method may be used to generate mutated, or altered, microorganisms for identification of microorganisms with increased production of hydrogen gas, relative to SGT-T4™, as a phenotype. Thus the method may comprise contacting a disclosed microorganism with a mutagen and then screening the treated microorganism for increased hydrogen gas production in comparison to SGT-T4™. Non-limiting examples of increased production include an increased rate of production over a given period of time and/or increased total gas production over a given period.

The disclosure also includes a method of genetically engineering a disclosed microorganism. In some embodiments, the method includes genetically engineering a disclosed microorganism, such as SGT-T4™, by transformation of the microorganism with the use of one or more DNA-, RNA- or PNA-based vehicles, such as a plasmid or a bacteriophage. Additional embodiments further include screening a transformed microorganism for increased hydrogen production rates and/or output.

Having now generally described the invention, the same will be more readily understood through reference to the following examples which are provided by way of illustration, and are not intended to be limiting of the disclosed invention, unless specified.

### EXAMPLES

### Example 1

### General

### Environmental Sampling

The SGT-T4™ microorganism of the disclosed invention was isolated from the dissected gut of a termite species found in the U.S.A. For isolation, the gut of the surface-sterilized and dissected termite was carefully removed under sterile conditions, minced and transferred into sterile basic growth medium (6 g Tryptone, 3 g yeast extract, 10 g glucose, 0.3 g MgSO₄, 0.02 g CaCl₂, 67 mM K₂HPO₄/NaH₂PO₄ buffer, pH 7.0 in 1 liter distilled water). Serial solutions of the dissolved gut homogenates were made in the basic growth media and then incubated under aerobic and anaerobic conditions at 30° C. for several days. Aliquots of test tubes showing bacterial growth were streaked onto the surface of selective agar plates containing growth media and incubated at 35° C. for one to three days in a humidified incubator.

### Growth Medium, Isolation and Cultivation

Single colonies of the plates grown under aerobic and anaerobic conditions were picked, re-inoculated in basic growth medium and restreaked on agar plates. Single colonies of these plates were picked and tested for hydrogen production as described below.

### Example 2

### Measurement of Gas and Hydrogen Production

Picked individual colonies were tested for total gas production using inverted Durham test tubes filled with either basic growth medium (10 ml) as described above or filled with other complex media (10 ml) as described in more detail in the examples below. Alternatively, total gas production of isolated colonies was detected using the BBL enterotube testing system. During this screening effort a microorganism, termed SGT-T4™, was discovered as a bacterium with the highest total gas production within a time period of less than 24 hours.

Hydrogen gas production of the microorganism was measured and achieved using following experimental set-up and incubation conditions. An aliquot (350 μl) of an over night culture of SGT-T4™ (grown in modified basic growth medium as described above) was inoculated into 50 ml of sterile complex medium 1 (14 g K₂HPO₄, 6 g KH₂PO₄, 5 g peptone, 2 g (NH₄)₂SO₄, 0.2 g MgSO₄×7H₂O, 1 ml trace element solution, 15 g glucose (or other carbon feedstock), pH 7.0 in 1 liter distilled water) and transferred into a 250 ml size fermentation vessel. After sealing off the fermentation vessel with a two-way inlet rubber stopper, the content of the vessel was flushed for 10 minutes with pure argon gas at a flow rate of 10 ml/min. The incubation vessel with the inoculated bacteria was placed in a shaking water bath or stirred fermentation platform and incubated at 37° C. Time-dependent generation of hydrogen gas in the vessel was monitored with the help of a linked fuel cell system (Hydro-genius™, HelioCentris, Berlin, Germany).

The hydrogen gas-induced increase in current and voltage at the fuel cell was recorded with the help of a fuel cell-connected amperemeter (DT 830B multimeter) and voltmeter (Fluke 10 multimeter). Alternatively, the time-dependent evolution of hydrogen gas of the inoculated bacteria was measured by a liquid-gas exchange method using an upside-down graduated measuring cylinder (250 ml) which was tube-connected with the incubation vessel. The liquid in the cylinder was a freshly prepared 15% NaOH solution which quantitatively (>97%) absorbed the CO₂ fraction of the evolved gas from the fermentation vessel. Therefore, the gas production rate measured by the graduated measuring cylinder was considered as the hydrogen gas (H₂) production and was standardized to ml H₂ evolved per hour per liter fermentation volume. Using both hydrogen production monitoring and measurement methods, the isolated microorganisms SGT-T4™ was found to be a rapid and high hydrogen gas producer. The discovered hydrogen producing microorganism SGT-T4™ was further characterized and its cultivation conditions optimized for maximum and long term hydrogen production under batch conditions.

### Example 3

### Biochemical Analysis and Identification

An isolated colony of SGT-T4™ was inoculated into basic growth medium and grown at 37° C. between 12-24 hours. Morphological examinations and cell counting were performed with a compound light microscope (Olympus, Japan) using the oil immersion method. Gram-staining, which was performed by the Hucker method, and motility testing using the semisoft agar medium method revealed that the high hydrogen gas producing microorganism is a gram-negative, motile, non-sporulating and non-capsulated short rods which grow under aerobic and anaerobic growth conditions (see ‘General Properties’ in Table 1).

TABLE 1
GENERAL PROPERTIES OF SGT-T4

| | | Growth Ability | |
|---|---|---|---|
| Strain | SGT-T4 | Aerobic | Yes |
| Gram Staining | Negative | Anaerobic | Yes |
| Shape | short rod | Tryptone-Yeast Water | Yes* |
| Motility | Motile | Tryptone-Yeast Water | Yes* |
| | | (+Glucose) | |
| Spore | Non-spore | Minimum Medium | No* |
| Capsule | No | Minimum Medium | Yes* |
| | | (+Glucose) | |

*= under aerobic growth conditions; 1% glucose as carbon source

Based upon further examined biochemical properties of the isolated microorganism using the BBL Enterotube II system (BD Diagnostics, U.S.A.) and individual biochemical tests (see Table 2), the biochemical profile of SGT-T4™ appeared most similar to the reported features of the enterobacterium Enterobacter aerogenes (see Bergey's Manual of Determinative Bacteriology; 9th edition).

TABLE 2
BIOCHEMICAL PROPERTIES OF SGT-T4

| | SGT-T4 ™ | SGT06-1 ™ | E.a.* |
|---|---|---|---|
| Strain | | | |
| Gram stain | − | − | − |
| Spore stain | − | − | − |
| Motility | + | + | + |
| Indole | − | − | − |
| Voges-Proskauer (Acetoin) | + | + | + |
| Methyl Red | − | − | − |
| Citrate | + | + | + |
| Gas (f. Glucose) | + | + | + |
| H₂S | − | − | − |
| Urease | + | − | − |
| Phenylalanine deaminase | − | − | − |
| Lysine decarboxylase | + | − | + |
| Ornithine decarboxylase | + | + | + |
| Oxidase | − | − | − |
| Catalase | + | + | + |
| Growth & Acid (aerobic)#: | | | |
| D-Glucose (plus gas) | + | + | + |
| D-Adonitol | + | + | + |
| L-Arabinose (plus gas) | + | + | + |
| Cellobiose (plus gas) | + | + | + |
| Dulcitol | − | − | − |
| Lactose | − | − | − |
| Sucrose (plus gas) | + | + | + |
| Maltose (plus gas) | + | + | + |
| D-Xylose (plus gas) | + | + | + |
| D-Mannose (plus gas) | + | + | + |
| D-Sorbitol (plus gas) | + | + | + |
| D-Mannitol (plus gas) | + | + | + |
| L-Rhamnose (plus gas) | + | + | + |
| D-Galactose (plus gas) | + | + | n/a |
| Glycerol | + | − | n/a |
| Starch | − | − | n/a |
| Cellulose | − | − | n/a |

*E.a. = Enterobacter aerogenes; information based on Bergey's Manual of Determinative Bacteriology’ (9th edition)
n/a = data not available in Bergey's Manual of Determinative Bacteriology
#concentration for all carbohydrates under investigation = 1.5%; results observed and recorded after 24 hours incubation time

Despite the similarities to the enterobacterium Enterobacter aerogenes, SGT-T4™ showed three major difference to the reported characteristics of Enterobacter aerogenes. First it did not grow well with lactose as feedstock and did not generate significant amounts of gas after 24 hours incubation in the growth media. Second, in variation to known Enterobacter aerogenes bacteria, SGT-T4™ was able to grow and to evolve gas with urea as sole nitrogen source in the medium. Without being bound by theory, and offered to improve the understanding of the disclosure, this is consistent with the bacterium being urease-positive. Third, SGT-T4™ shows minimum spontaneous sedimentation in growth media after prolonged non-agitated incubation when directly compared with the commercially available bacterium Enterobacter aerogenes ATCC13048.

When directly compared with the biochemical properties of Enterobacter sp. SGT06-1™ (another hydrogen producing microorganism), SGT-T4™ showed three significant differences. First, SGT-T4™ but not SGT06-1™ was able to grow in pure glycerol as carbon feedstock and to generate gas. Second, in variation to the SGT06-1™ bacteria, SGT-T4™ was able to grow and to evolve gas with urea as sole nitrogen source in the medium. Finally, SGT-T4™,but not SGT06-1™, is a lysine decarboxylase enzyme-positive microorganism.

### Example 4

### Total Gas Production of SGT-T4 with Glucose and Zeolite Effect

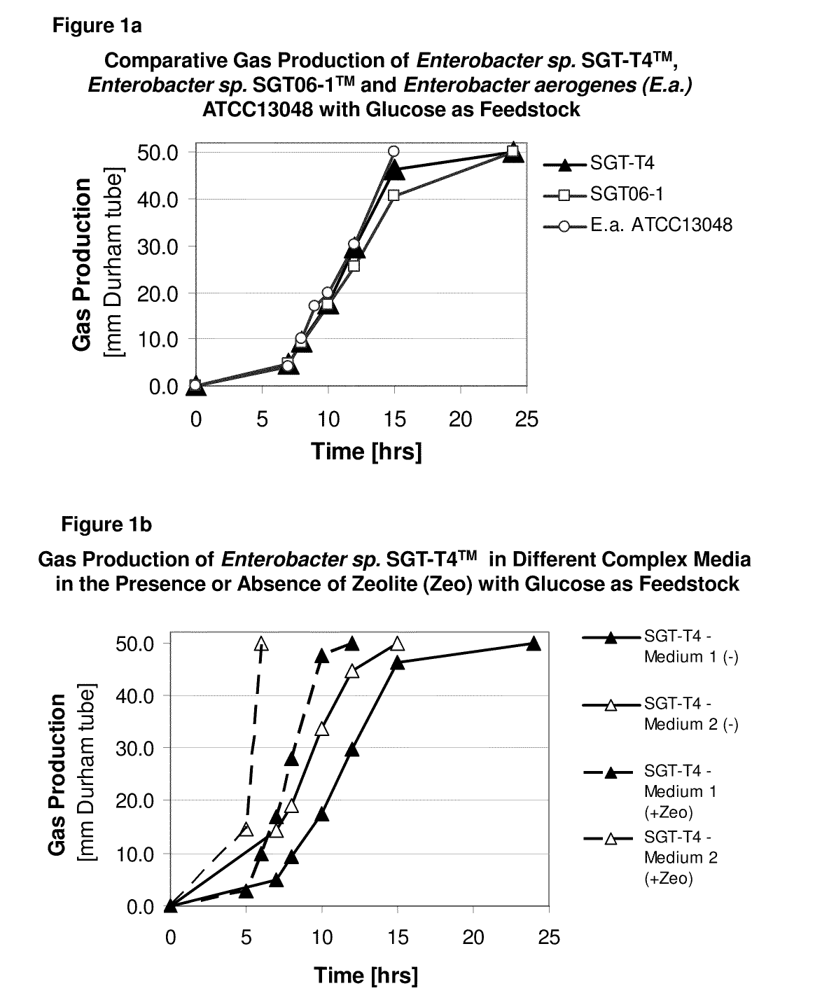

The high evolution of gas from SGT-T4™, especially under anaerobic conditions, as indicated by the BBL Enterotube analysis system was further analyzed. Direct comparisons were made of the total gas production from SGT-T4™ with Enterobacter sp. SGT06-1™ and Enterobacter aerogenes ATCC 13048 (E.a.), both known high hydrogen gas producing bacteria. Each of the three microorganisms was inoculated in Durham test tubes filled with 10 ml of peptone-glucose (PG) medium (14 g K₂HPO₄; 6 g KH₂PO₄; 5 g peptone; 2 g (NH₄)₂SO₄; 0.2 g MgSO₄×2H₂O; 15 g glucose per 1 liter). The bacteria were incubated at 37° C. in an incubator and monitored for the evolution of gas at defined time intervals over 24 hours. The results of this set of experiments, which are shown in FIG. 1a (plotted as mm total gas accumulation in the inverted Durham tubes) confirm the high gas production from SGT-T4™. The total gas production of SGT-T4™ with glucose as carbon feedstock showed no significant difference to that of SGT06-1™ or of Enterobacter aerogenes ATCC 13048. Similar high gas production under anaerobic conditions was observed by incubating the newly isolated microorganism in Durham test tubes filled with glucose minimum (synthetic) medium (results not shown).

Next a series of experiments was conducted to find conditions which further increase the high hydrogen production rate of the isolated microorganism. For this, SGT-T4™ was inoculated in Durham test tubes filled with 10 ml of either peptone-glucose (PG) medium (14 g K₂HPO₄; 6 g KH₂PO₄; 5 g peptone; 2 g (NH₄)₂SO₄; 0.2 g MgSO₄×7H₂O; 21 mg CaCl₂×2H₂O; 20 g glucose per 1 liter) or tryptone-yeast-glucose (TYG) medium (7 g K₂HPO₄; 5.5 g KH₂PO₄; 5 g tryptone; 5 g yeast extract; 1 g (NH₄)₂SO₄; 0.25 g MgSO₄×7H₂O; 0.12 g Na₂MoO₄×2H₂O; 2 mg nicotinic acid; 0.172 mg Na₂SeO₃; 0.02 mg NiCl₂; 2 mg MnCl₂×4H₂O; 20 g glucose per 1 liter) in the presence or absence of 2.5% of the strongly absorbent aluminosilicate zeolite (Zeo). The bacteria were incubated at 37° C. in an incubator and monitored for the evolution of gas at defined time intervals over 24 hours. The results of this set of experiments, which are shown in FIG. 1b, show that the high gas production of SGT-T4™ in PG medium is further increased in TYG medium and that the gas production in both media is significantly increased in the presence of 2.5% zeolite in the growth medium.

The calculated total gas production rate of SGT-T4™ was about 43 percent (43%) higher when it is cultivated in TYG medium (261 ml gas/hour per liter in PG medium versus 374 ml gas/hour per liter in TYG medium). Presence of zeolite in the growth medium increased the gas production in PG medium by a factor of 1.87 (87%) and in TYG medium to more than 8.6 times (865%). The effect of zeolite on the gas production of SGT-T4 in TYG medium is dramatic and is the highest percent gas production rate increase ever reported in the published literature and known to the present inventors.

### Example 5

### Hydrogen Production of SGT-T4 with Glucose and Zeolite Effect

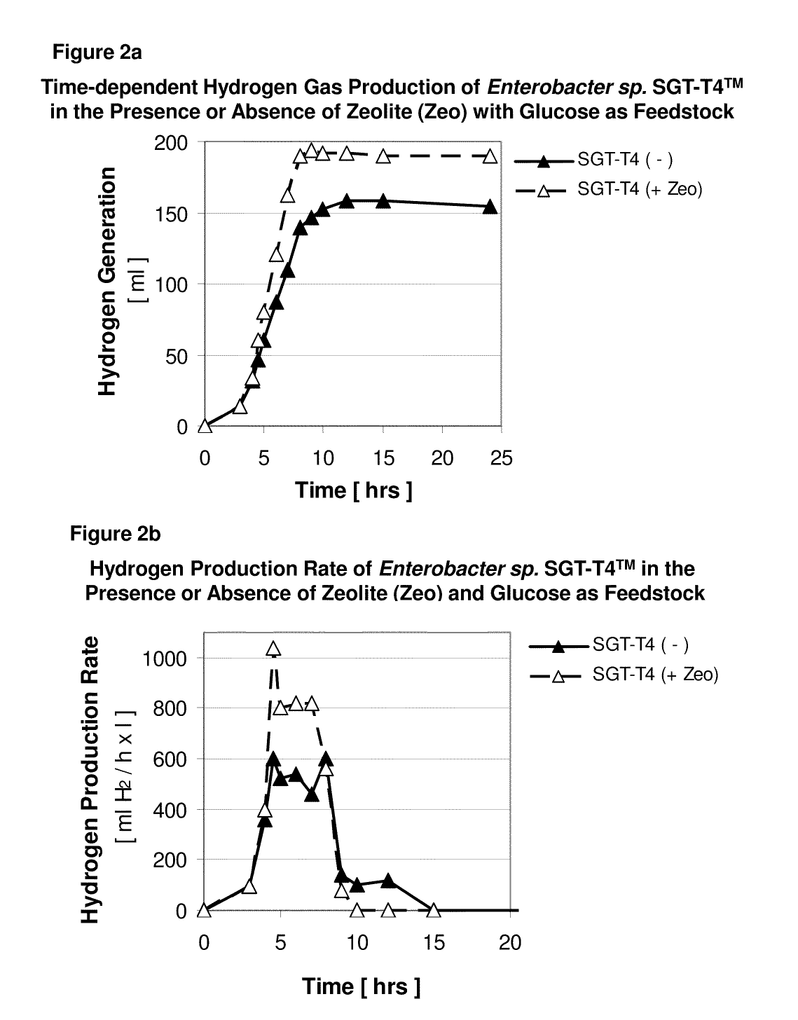

The hydrogen production rate of SGT-T4™ with glucose as carbon feedstock was measured in the presence or absence of 2.5% zeolite in the medium. For this a liquid-gas exchange method was used which consisted of an upside-down graduated cylinder filled with a 15% NaOH solution that was tube-connected with the gas outlet of the bio-reactor containing the cultivated bacterium under investigation. Due to the absorption of carbon dioxide—the only concomitantly released gas by the isolated bacterium SGT-T4™ by the NaOH solution in the inverted cylinder, the gas production rate measured with the help of a graduated cylinder was considered to be the hydrogen evolution rate of the bacteria under investigation. Using this method and incubating the bacteria under batch conditions in 50 ml tryptone-yeast medium at a temperature of 37° C. and with 2% glucose as feedstock, SGT-T4 evolved 154 ml of hydrogen gas (H₂) in 24 hours (FIG. 2a) and the maximum hydrogen production rate of SGT-T4™ was measured to be 600 ml hydrogen gas (H₂) produced per hour per liter (ml/h×l) (FIG. 2b).

This rate is the highest hydrogen production rate under batch conditions ever reported for a hydrogen producing microbe with glucose as feedstock (for comparison see Table 3 below) and exceeds the high total gas and hydrogen production rates reported by Taguchi et al. (U.S. Pat. No. 5,350,692) for the anaerobic microorganisms AM21B and AM37 in peptone-yeast glucose (PYG) medium. The high hydrogen production rate of SGT-T4 with glucose as feedstock was increased about 77 percent (77%) in the presence of 2.5% zeolite in the growth medium. At about 4.5 hours incubation time a hydrogen production rate of more than 1 liter of H₂ per hour per liter (1,060 ml H₂/h×l) was measured (FIG. 2b). The very high hydrogen gas production rate of the microorganism SGT-T4™ under the chosen incubation conditions was further confirmed by detecting the generated hydrogen gas with the help of a vessel-connected and calibrated fuel cell system (Hydro-genius™, HeliCentris, Berlin, Germany) (Data not shown).

TABLE 3
COMPARATIVE HYDROGEN PRODUCTION RATES

| | Hydrogen Gas Rate | |
|---|---|---|
| Species* | (ml H₂/h × 1) | References |
| Enterobacter sp. SGT-T4 ™ | 600 | Schmid E. et al. |
| | 1,060⁺ | (unpublished results) |
| Enterobacter sp. SGT06-1 ™ | 460⁺ | Schmid E. et al. |
| | | (unpublished results) |
| Klebsiella oxytoca | 87.5 | Minnan L. et al., Res. Microbiol. |
| | | 156(1): 76-81 (2005) |
| Citrobacter freundii | 90 | Kumar G. R. et al., Indian J. Exp. |
| | | Biol. 27(9): 824-825 (1989) |
| Enterobacter aerogenes | 253 | Tanisho S. et al., J. Chem. Eng. |
| E.82005 | | (Japan) 16: 529ff (1983); |
| | | Tanisho S. et al., Int. J. Hydrogen |
| | | Energy 12: 623ff (1987) |
| Enterobacter aerogenes | 372 | Ito T., et al., J. Biosci. Bioeng. |
| HU-101 | | 97(4): 227-232 (2004) |
| Enterobacter aerogenes | 120 | Yokoi H. et al., J. Ferment. |
| | | Bioeng. 80: 571ff (1995) |
| Clostridium beijerinckii | 210 | Taguchi F. et al., U.S. |
| | | Pat. No. 5,350,692 (Sep. 27, |
| | | 1994) |
| Clostridium butyricum | 75 | Ogino H. et al., Biotechnol. Prog. |
| | | 21(6): 1786-1788 (2005) |
| Mixed Anaerobes | 230 | Iyer P. et al., Appl. Microbiol. |
| | | Biotechnol 66: 166-173 (2004) |
| Mixed bacterial cultures | 74.7 | Van Ginkel S. et al., Environ. Sci. |
| | | Technol. 35(24): 4726-4730 |
| | | (2001) |
| Thermotoga elfii | 125 | Van Niel E. W. J. et al., Hydrogen |
| | | Energy 27: 1391-1398 (2002) |
| Caldicellulosiruptor- | 250 | Van Niel E. W. J. et al., Hydrogen |
| saccharolyticus | | Energy 27: 1391-1398 (2002) |
| Caldicellulosiruptor- | 250 | Kadar Z. et al., Appl. Biochem. |
| saccharolyticus | | Biotechnol. 113-116: 497-508 |
| | | (2004) |
| Thermotoga neapolitana | 460 | Van Ooteghem S. A. et al., Appl. |
| | | Biochem. Biotechnol. 98-100: |
| | | 177-189 (2002) |

*all microorganisms cultivated in batch cultures in the presence of glucose
⁺cultivated in the presence of 2.5% natural zeolite (clinoptilolite) in the medium

SGT-T4™ shows rapid growth and reaches high optical densities not only in the presence of the carbohydrate glucose, but also when cultivated in the presence of other carbohydrate feedstock, such as sucrose, cellobiose, maltose, xylose, arabinose, rhamnose, galactose, sorbitol, mannitol, and mannose (data not shown).

### Example 6

### Total Gas Production of SGT-T4™ with Different Carbon Feedstock

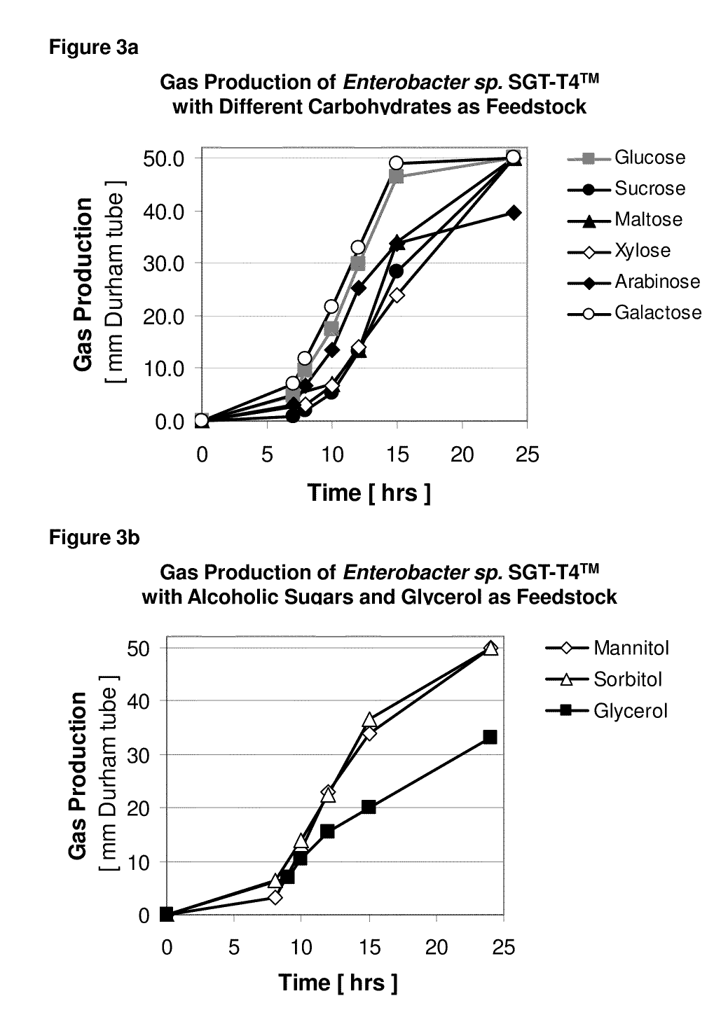

The gas production capacity of SGT-T4™ was tested in the presence of carbohydrates and carbon feedstock other than glucose. This tested whether SGT-T4™ is metabolically versatile and is capable of generating comparatively high amounts of gas in the presence of important biomass-derived carbon compounds as feedstock. The present inventors were especially interested whether the disaccharides sucrose and maltose, the hemicellulosics-derived carbohydrates xylose, arabinose and galactose, the alcoholic sugars mannitol and sorbitol, as well as the phospholipid and fat-derived carbon compound glycerol also serve as suitable feedstock for the isolated microorganism. As shown in FIG. 3a, SGT-T4™ generates high amounts of gas with glucose as feedstock (grey squares), and also when cultured in the presence of maltose, sucrose, arabinose, xylose and galactose. It is of interest that the time-dependent gas production of SGT-T4™ shows a distinctive prolonged lag phase with maltose, sucrose, xylose and arabinose as feedstock when directly compared with glucose, while SGT-T4 responded with an even stronger gas production than with glucose in the presence of the monosaccharide galactose as carbon source. This finding, where the isolated bacterium SGT-T4™ is able to generate high amounts of gas from more than monosaccharide glucose as feedstock, but also from the important plant-derived disaccharides sucrose and maltose, as well as in the presence of key hemicellulosics sugars, such as xylose and arabinose, is of high commercial value. It allows simplified and cost saving future industrial scale hydrogen production from traditionally high sucrose-containing wastes, such as bagasse and food industry wastes, maltose-containing waste streams, such as brewery wastes, and from materials abundant in hemicellulose, such as plant matter.

### Example 7

### Gas Production from Alcoholic Sugars and Glycerol

Yet another important set of studies conducted was the capability of SGT-T4™ to generate high quantities of gas in the presence of the alcoholic sugars mannitol and sorbitol, and when cultured in the presence of the tertiary alcohol glycerol. As shown in FIG. 2b, SGT-T4™ generates very high amounts of gas in the presence of the alcoholic sugars mannitol and sorbitol in the growth medium within 24 hours incubation time. The gas production of SGT-T4™ with glycerol as carbon feedstock was not as high as with mannitol or sorbitol under the chosen incubation conditions. The observation whereas SGT-T4™ is capable of generating high amounts of hydrogen gas from the alcoholic sugars mannitol and sorbitol, makes it a potentially attractive microorganism for future industrial scale generation of hydrogen energy from sources and waste streams rich in these alcoholic sugars, such as brown algae and nutritional industry.

### Example 8

### Increased Gas Production of SGT-T4 with Glycerol or Crude Bio-Diesel Production Waste in the Presence of Zeolite

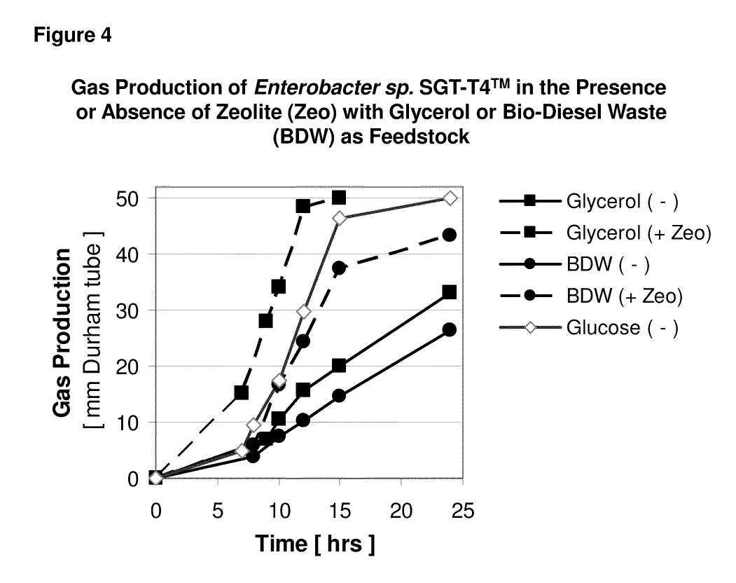

A set of studies was conducted to study the effect of an aluminosilicate mineral on the gas production of SGT-T4™ when cultured in the presence of the tertiary alcohol glycerol. As shown in FIG. 4, the low gas production rate of SGT-T4™ with glycerol (300 mM) and without zeolite in the growth medium (91 ml gas per hour per liter) was increased 2.4 times when defined amounts of zeolite (2.5%) were present in the growth medium during the incubations. In the presence of zeolite the gas production rate of SGT-T4™ increased to about 220 ml gas per hour per liter. Since bio-diesel waste contain high (>40%) concentrations of glycerol, the authors of this disclosure conducted experiments to test whether SGT-T4™ is capable to generate high amounts of gas when cultivated in the presence of a defined volume of glycerol-containing crude bio-diesel waste (BDW) collected from a local bio-diesel processor. For this SGT-T4™ was incubated in 10 ml tryptone-yeast growth medium containing 0.75 ml of crude BDW in the presence or absence of defined amounts of zeolite. As shown in FIG. 4, the low total gas production rate of SGT-T4™ with bio-diesel waste (BDW) and without zeolite in the growth medium of about 53 ml gas per hour per liter) was increased more than 2.7 times when defined amounts of zeolite (2.5%) were present in the growth medium during the incubations. In the presence of zeolite the gas production rate of SGT-T4™ increased to about 148 ml gas per hour per liter, which was almost as high as the gas evolution rate observed with glucose as carbon feedstock (173 ml gas per hour per liter; in the absence of zeolite). The observation that SGT-T4™ is capable of generating high amounts of gas from the tertiary alcohol glycerol and also from high glycerol-containing bio-diesel waste in the presence of zeolite mineral in the growth medium makes it a potentially attractive microorganism for industrial scale hydrogen production for the rapidly developing bio-diesel processing industry.

### Example 9

### Increased Hydrogen Gas Production of SGT-T4 with Glycerol or Crude Bio-Diesel Production Waste in the Presence of Zeolite

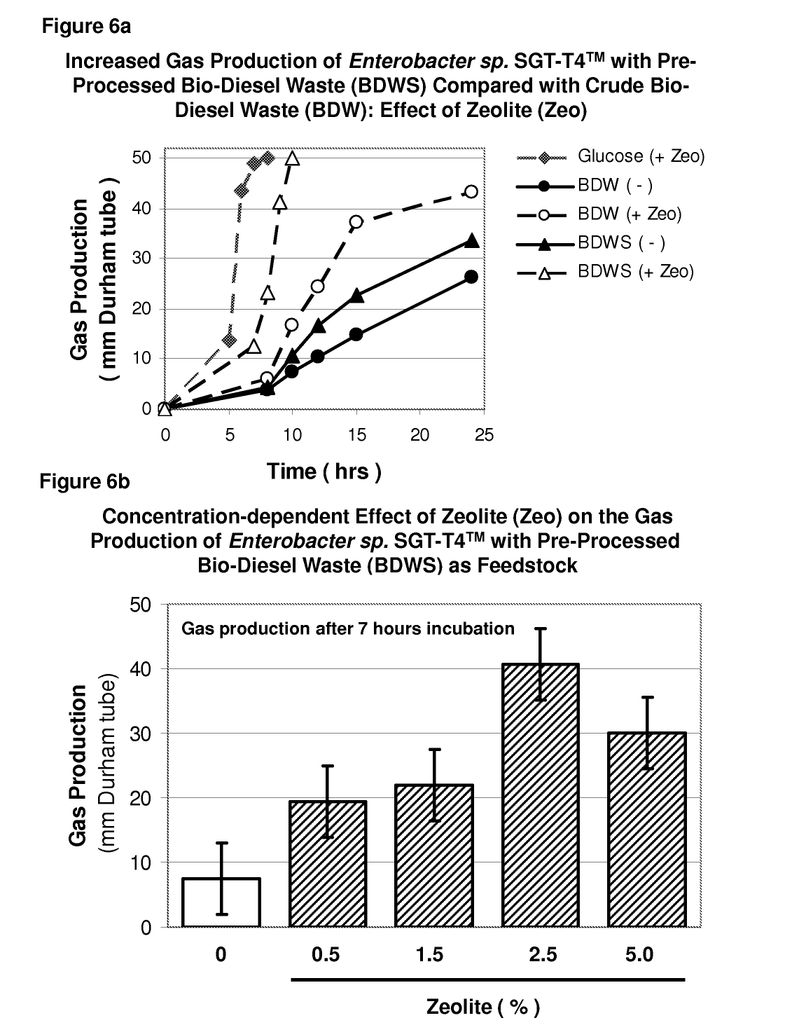

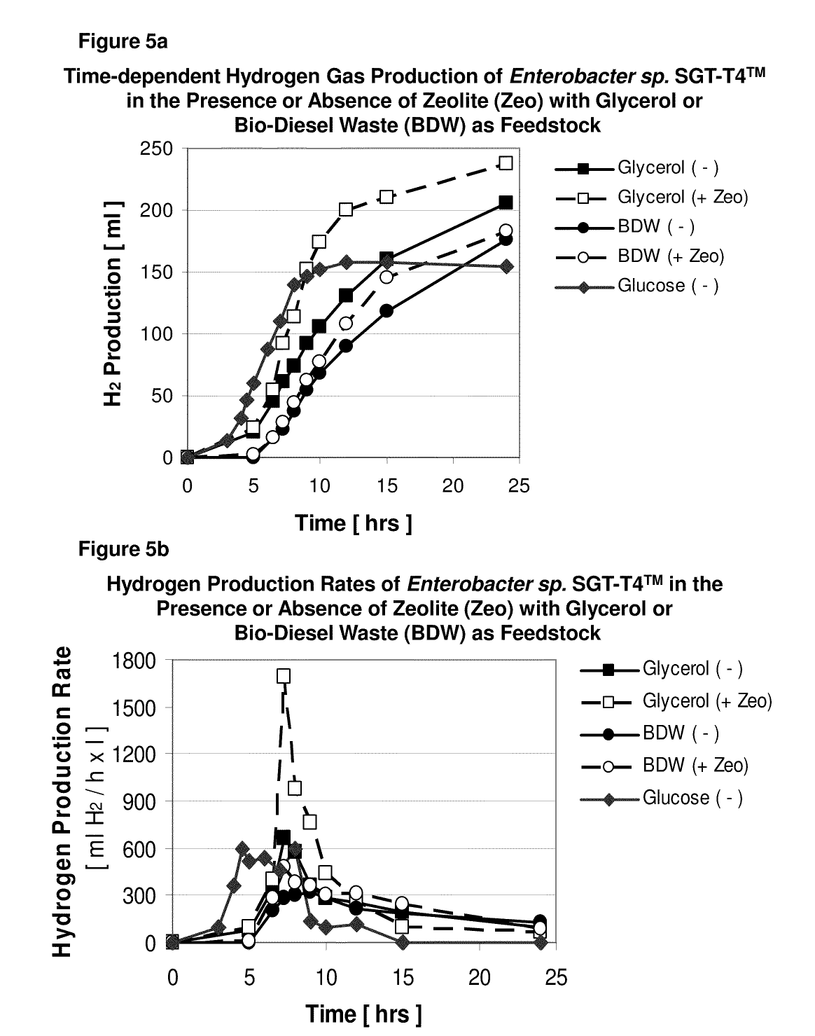

The amount of hydrogen gas evolved over time and the hydrogen production rate of SGT-T4™ with glycerol or crude bio-diesel waste (BDW) as carbon feedstock was examined in the presence or absence of zeolite in the growth medium. For this, the same liquid-gas exchange method was used as described in more detail in Example 5. It consisted of an upside-down graduated cylinder filled with a 15% NaOH solution that was tube-connected with one of the outlets of the bio-reactor. The bio-reactor was filled with 50 ml of growth medium containing the cultivated bacterium under investigation and where indicated in FIG. 5 with 2.5% zeolite material. Due to the absorption of carbon dioxide—the only concomitantly released gas by the isolated bacterium SGT-T4™—by the NaOH solution in the inverted graduated cylinder, the gas production rate measured with the use of a graduated cylinder was considered to be the hydrogen evolution rate of the bacteria under investigation. As shown in FIG. 5a, using this method and incubating the bacteria under batch conditions in 50 ml tryptone-yeast medium at a temperature of 37° C. and with pure industrial glycerol as feedstock, SGT-T4 evolved 206 ml of hydrogen gas (H₂) in 24 hours in the absence of zeolite in the growth medium. The volume of hydrogen gas evolved by SGT-T4™ after 24 hour incubation increased more than 15% to 237 ml in the presence of 2.5% zeolite in the growth medium. The calculated maximum hydrogen production rate of SGT-T4™ with glycerol as feedstock occurred at around 7 hours incubation time and was 667 ml hydrogen gas (H₂) produced per hour per liter (667 ml/h×l) in the absence of zeolite (FIG. 5b). This remarkably high rate further increased to 1,689 ml hydrogen gas (H₂) produced per hour per liter (1,689 ml/h×l) when 2.5% zeolite was present in the growth medium (FIG. 5b) accounting for a rate increase of more than 250%.

This high rate of SGT-T4™ with glycerol as feedstock and in the presence of defined amounts of zeolite in the growth medium is the highest hydrogen production rate under batch conditions ever reported for a hydrogen producing microbe with this feedstock. For comparison, the volumes of hydrogen gas evolved by SGT-T4™ from 300 mM glycerol in the presence (237 ml; 209 mM) or absence (206 ml; 182 mM) of zeolite within 24 hours both exceed the reported H₂ volume of 93 ml (53 mM) generated by Enterobacter aerogenes HU-101 in 24 hours in the presence of 110 mM glycerol as feedstock [Ito T., et al.; J. Bioscience & Bioengineering 100(3): 260-265 (2005)]. A 300 ml culture of Enterobacter aerogenes NBRC12010 was recently reported to generate about 265 ml (40 mM) H₂ from glycerol (110 mM) in 24 hours under chemostat conditions (see Sakai S. & Yagashita T.; Biotechnology & Bioengineering 98(2): 340-348 (2007)). Based on these comparisons, the present inventors believe that SGT-T4™ is an ideal candidate microorganism for conversion of glycerol and glycerol-containing waste streams into clean hydrogen energy under favorably high production rate. To test this, SGT-T4™ was incubated in 50 ml tryptone-yeast growth media together with 3.8 ml of collected bio-diesel waste in the presence or absence of zeolite.

As shown in FIG. 5a, using the earlier described liquid-gas exchange method and incubating SGT-T4™ under batch conditions in 50 ml tryptone-yeast medium at a temperature of 37° C. and with crude bio-diesel waste (BDW) as feedstock, SGT-T4 evolved 176 ml of hydrogen gas (H₂) in 24 hours in the absence of zeolite in the growth medium. The volume of hydrogen gas evolved by SGT-T4™ after 24 hour incubation increased to 183 ml in the presence of 2.5% zeolite in the growth medium. The calculated maximum hydrogen production rate of SGT-T4™ with BDW as feedstock occurred between 7 and 8 hours incubation time and was 320 ml hydrogen gas (H₂) produced per hour per liter (320 ml/h×l) in the absence of zeolite (FIG. 5b). This rate further increased to almost 500 ml hydrogen gas (H₂) produced per hour per liter (480 ml/h×l) when 2.5% zeolite was present in the growth medium (FIG. 5b) accounting to a rate increase of about 50%.

### Example 10

### Increased Hydrogen Gas Production of SGT-T4 with Pre-Processed Bio-Diesel Waste in the Presence of Zeolite

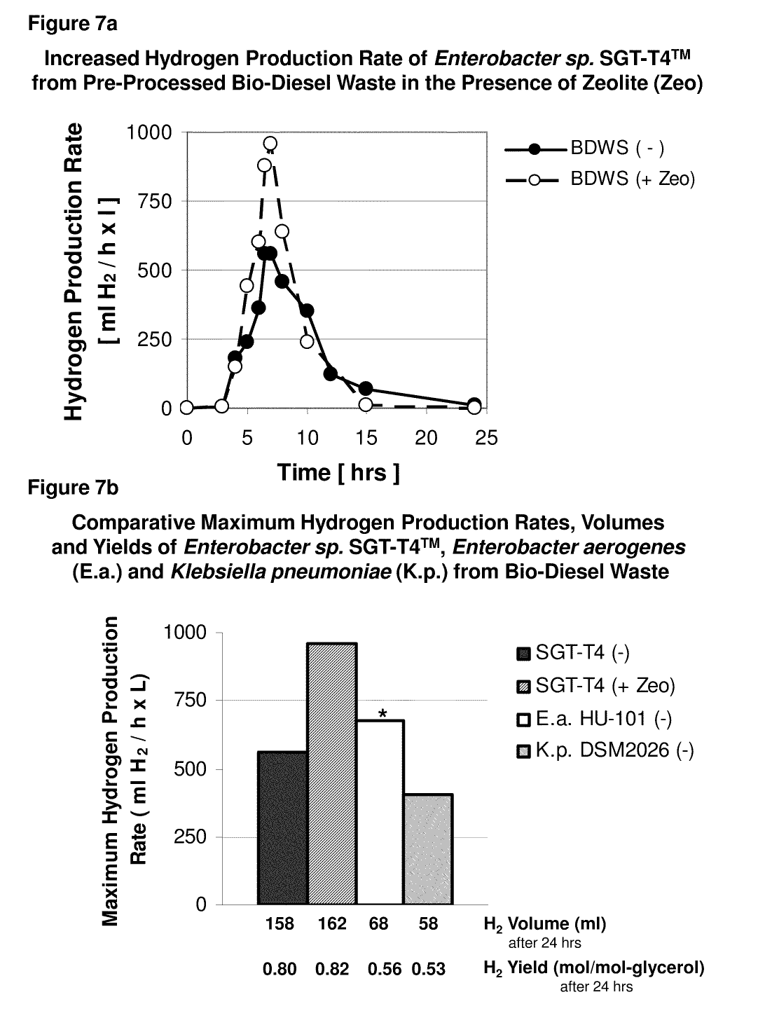

The amount of hydrogen gas evolved over time, hydrogen production rate and hydrogen production yield of SGT-T4™ with pre-processed bio-diesel waste (BDWS) as carbon feedstock was examined in the presence or absence of zeolite in the growth medium. Pre-processed bio-diesel waste solution (BDWS) was freshly prepared before the experiment by dissolving 40 ml of pre-cleared bio-diesel waste (BDW) in 460 ml of sterile distilled water followed by pH-neutralization of the alkaline pH of the BDWS with a 6N HCl solution. In this example, the same liquid-gas exchange method was used as described in more detail in Examples 5 and 9 to measure the generation of hydrogen gas by SGT-T4™. It consisted of an upside-down graduated cylinder filled with a 15% NaOH solution that was tube-connected with one of the outlets of the bio-reactor. The bio-reactor was filled with 25 ml of complex growth medium and 25 ml of BDWS containing the cultivated bacterium under investigation and where indicated in FIG. 7a with 2.5% zeolite material. Due to the absorption of carbon dioxide—the only concomitantly released gas by the isolated bacterium SGT-T4™—by the NaOH solution in the inverted cylinder, the gas production rate measured with the help of the graduated cylinder was considered to be the hydrogen evolution rate of the bacteria under investigation.

As shown in FIG. 7a, using this method and incubating the bacteria under batch conditions in 25 ml tryptone-yeast medium at a temperature of 37° C. and with 25 ml BDWS as feedstock, SGT-T4 evolved 158 ml of hydrogen gas (H₂) in 24 hours in the absence of zeolite in the growth medium. The calculated maximum hydrogen production rate of SGT-T4™ with BDWS as feedstock and in the absence of zeolite in the reaction vessel occurred at around 7 hours incubation time and was 560 ml hydrogen gas (H₂) produced per hour per liter (560 ml/h×l). Under identical incubation conditions, this high hydrogen production rate further increased to 960 ml hydrogen gas (H₂) produced per hour per liter (960 ml/h×l) when 2.5% zeolite was present in the growth medium accounting to a rate increase of more than 70%. The measured hydrogen production rate of SGT-T4™ with pre-processed bio-diesel waste solution (BDWS) as feedstock and in the presence of 2.5% zeolite in the growth medium is the highest hydrogen production rate under batch conditions reported for a hydrogen producing microbe with bio-diesel refinery waste as feedstock to date. For comparison, the hydrogen production rate (960 ml H₂/h×l), yield (0.82) and 24 hour volume of hydrogen gas (162 ml) evolved by SGT-T4™ from BDWS in the presence of zeolite exceeds the reported hydrogen production rates, yields and 24 hour volumes of the known hydrogen producing microbes Enterobacter aerogenes HU-101 (678 ml H₂/h×l, 0.56, 68 ml) (see Ito T., et al.; J. Bioscience & Bioengineering 100(3): 260-265 (2005)) and Klebsiella pneumoniae DSM2026 (402 ml H₂/h×l, 0.53, 58 ml) [Liu F. & Fang B.; Biotechnol. J. 2(3): 374-380 (2007)] from bio-diesel waste as feedstock (FIG. 7b). Based on these comparisons, the authors of this disclosure believe that SGT-T4™ is an ideal candidate microorganism for economical conversion of glycerol-containing waste streams, most prominently bio-diesel waste refinery waste, into clean hydrogen energy under favorably high production rate conditions.

### Example 11

### Comparison of the Sedimentation Behavior of SGT-T4™ with Other Enterobacteria

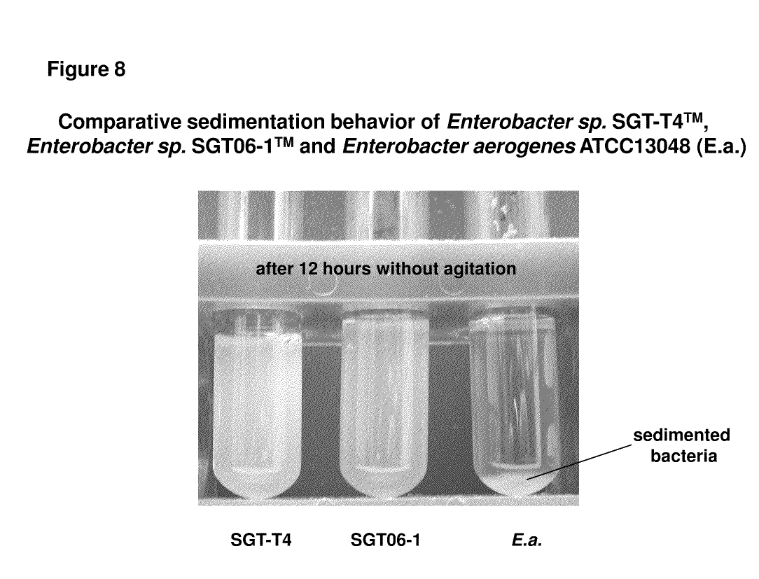

During comparative functional studies with the isolated microorganism SGT-T4™ and commercially available enterobacteria, such as the biochemically most closely related Enterobacter aerogenes species, the present inventors observed a strikingly different sedimentation behavior of SGT-T4™ in the growth media used in these studies. As shown in FIG. 8, when directly compared with Enterobacter aerogenes ATCC13048, SGT-T4™ showed no signs of sedimentation and no bacterial cell pellet formed at the bottom of the test tube after 12 hours incubation in the absence of test tube agitation during this incubation period. That significant difference between the sedimentation behavior of SGT-T4™ and a commercially available Enterobacter aerogenes species is indicative of significant differences in cell morphologies and/or motility. This low sedimentation behavior of SGT-T4™ might be beneficial in large scale bio-reactor environments where continuous stirring of the media is usually required which is a significant operation cost factor.

### Example 12

### PCR and 16S rRNA Gene Sequence Analysis

To better assign microorganism SGT-T4™ to the genus Enterobacter aerogenes within the enterobacteria family, molecular biological methods were used to identify the isolate by 16S-rRNA gene sequence analysis. For direct comparison and for serving as an internal control of the following procedure, PCR with DNA isolated from Enterobacter aerogenes (ATCC13048 strain) was used as an internal standard and control of the applied method (data not shown).

PCR-dependent 16S rRNA gene sequence analysis was carried out as follows. Isolates were grown in basic growth medium A for 20-24 hours at 37° C. and genomic DNA was isolated from pellets of collected bacterial cells (1 ml) using the Qiagen silica spin column method. A fragment of about 700 bp of the 16S rRNA gene of the isolated genomic DNA of SGT-T4™ was amplified by PCR using a designed “universal” 16S rRNA primer pair (SGT-UNI04fw3 and SGT-UNI04rv2 (see Table 4 below). SGT-UNI04fw3 and SGT-UNI04rv2 recognize highly conserved nucleotide sequences of the GenBank-deposited 16S rDNA sequence (nucleotide 140-160; nucleotide 824-841) of Citrobacter freundii ATCC 29935 (gi: 174064), and span a hypervariable region of the C. freundii 16S rRNA gene.

TABLE 4
USED PCR PRIMER FOR 16S rDNA ANALYSIS OF SGT-T4™

| | |
|---|---|
| SGT-UNI04-fw3 | 5′- TGGAGGGGGATAACTACTGG -3′ |
| | (SEQ ID No: 2) |
| SGT-UNI04-rv2 | 5′- GGCACAACCTCCAAGTCG -3′ |
| | (SEQ ID No: 3) |

Twenty picomoles of forward primer (SGT-UNI04-fw3) and reverse primer (SGT-UNI04-rv2) were used in the PCR reaction. The PCR reaction mixture further contained 0.5 units Taq polymerase (Invitrogen), 500 ng of genomic DNA, 0.1 mmol/l of each nucleotide (dNTPs) and 1.5 mM MgCl₂, in a total volume of 20 μl. A fragment of the 16S rRNA gene was amplified after 35 cycles in an automated thermal cycler (Mycycler, BioRad, Inc., CA) using following temperature profile: (4 min at 95° C.; (30s at 95° C., 30s at 53° C., 2 min at 72° C.)₃₅ₓ; 5 min at 72° C.).

After separation by low melting agarose gel electrophoresis, the 16S-rRNA PCR product was excised and purified with use of the Qiagen gel purification kit. The base sequence of the purified 16S rRNA gene segment was determined by using the Tag Dye Deoxy Terminator Cycle Sequencing method (Seqxcel Inc., San Diego, Calif.) and compared with the nucleotide sequences deposited with the NCBI (National Center for Biological Information) database (all GenBank+EMBL+DDBJ+PDB sequences). A comparative analysis of the retrieved 671 base sequence (see Table 5) of SGT-T4™ was done with the GenBank database using NCBI BLAST (blastn & MegaBlast). It revealed that SGT-T4™ is related to gram-negative bacteria showing highest sequence similarity to members of the enterobacteriaceae family. The four top scoring sequence similarities reported for the submitted 16S rRNA gene sequences of the following databank-deposited microorganisms are listed below (rankings based on lowest Expect (E) values and highest maximum score):

1. Uncultured bacterium clone SJTU_B_14_72
(gi: 126113270; Accession #: EF402955.1)

| | | |
|---|---|---|
| Maximum Score: 756 | E Value = 0.0 | Max. Identity: 87% |

2. Enterobacter sp. DAP21
(gi: 163931346; Accession #: EU302846.1)

| | | |
|---|---|---|
| Maximum Score: 754 | E Value = 0.0 | Max. Identity: 87% |

3. Uncultured Enterobacteriaceae bacterium clone M7-54
(gi: 175941128; Accession #: EU530476.1)

| | | |
|---|---|---|
| Maximum Score: 752 | E Value = 0 | Max. Identity: 87% |

4. Uncultured Enterobacteriaceae bacterium clone M7-52
(gi: 175941126; Accession #: EU530474.1)

| | | |
|---|---|---|
| Maximum Score: 752 | E Value = 0.0 | Max. Identity: 87% |

Of the 100 reported most significant matches with the isolated 16S rDNA fragment, 27 out of 100 were enterbacteria species, 17/100 were uncultured enterobacteriaceae bacteria and 38/100 of the sequence homologies were reported for uncultured bacteria. Summarized, genomic DNA was isolated from SGT-T4™, and the base sequence has been successfully analyzed with an obtained 16S rDNA fragment. The microorganism SGT-T4™ is believed to belong to the enterobacteriaceae family based on this sequence analysis. Because the level of 16S rDNA gene identity with their closest taxonomically named relatives was less than 88%, and due to observed differences in urea utilization, lactose metabolism and motility between microorganism SGT-T4™ and Enterobacter aerogenes species, the isolated microorganism is believed to be an Enterobacter and perhaps represents a new species based on the presented unique biochemical and genetic features. Via the 16S rDNA gene analysis, which closely related the isolated microorganism to the enterobacterial species Enterobacter sp. DAP21, the microorganism is named Enterobacter sp. SCT-T4™ for further reference and preliminary classification. The isolated microorganism Enterobacter sp. SGT-T4™ was deposited with the American Type Tissue Collection (ATCC) on Apr. 10, 2008 with accession no. ATCC No: PTA-9150.

TABLE 5

BASE SEQUENCE OF 16S rRNA GENE FRAGMENT OF
SGT-T4™ (SEQ TD No: 1)

CACATCGCAT ACGTCGCAGA CCAAAGTGGG GGACCTTCGG
GCCTCATGCC ATCAGATGTG CCCAGATGGG ATTAGCTAGT
AGGTGGGGTA ATGGCTCACC TAGGCGACGA TCCCTAGCTG
ATGACCAGCC ACACTGGAAC TGATACACGG TCCAGACTCC
TACGGGAGGC AGCAGTGGGG AATATTGATT TATGGGCGCA
AGCCTGATGC AGCCATGCCG CGTGTATGAA CAAGGCCTTC
CGATTGTAAA TTGCTTTCTC CGAATAGGAA GGCCTGCTGG
TTAATAACCT TGCGGATTGA CTTTACTCGC AAACGAAGCA
CCGGCTAACT CCGTGCCTTA AGCCCTTCCT CCTCGGAGGG
TGCACTTTTT AATCCGAATT ACTGGTTCTT AAGCGCACGC
TGGCTGCCTG TCGCTTGCGA TGTGAAATCC CCGGGCTCCA
CCTGGGAACT GCATTCGAAA CTGGACCGCT AGAGTCTTGT
AGAGGGGGGT GGAATTCCTC GTGTACCGGT GAAATGCGTA
CAGATCTGGA AGAATACCCC CCACCAAGGC GGCCCCCTGG
ACAAAGACTG ACTCTCAGGT GCAAAACCGT GGGGAGCCCA
CTTGATTATA TACCCTGGTA GTCCACTCCG CTACCGATGT
CAACTTGATT CCCCCCTCCA A (671 BP)

### BIBLIOGRAPHY

U.S. Patents:

| | | | |
|---|---|---|---|
| 2,429,589 | October, 1947 | Wiley | 435/167. |
| 3,383,309 | May, 1968 | Chandler | 48/197. |
| 3,711,392 | January, 1973 | Metzger | 435/167. |
| 3,764,475 | October, 1973 | Mandels et al. | 435/209. |
| 4,480,035 | October, 1984 | Roychowdhury | 435/168. |
| 5,350,692 | September, 1994 | Taguchi et al. | 435/252.7 |
| 6,887,692 | December, 2002 | Paterek J R | 438/168. |
| 6,860,996 | March 2005 | Noike et al. | 210/603. |
| 6,942,998 | September, 2005 | Ooteghem | 435/168. |
| 11/829,599 | July 2007 | Schmid et al. | |

All references cited herein, including patents, patent applications, and publications, are hereby incorporated by reference in their entireties, whether previously specifically incorporated or not.

Having now fully described the inventive subject matter, it will be appreciated by those skilled in the art that the same can be performed within a wide range of equivalent parameters, concentrations, and conditions without departing from the spirit and scope of the disclosure and without undue experimentation.

While this disclosure has been described in connection with specific embodiments thereof, it will be understood that it is capable of further modifications. This application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

What is claimed is:

1. An isolated, hydrogen producing microorganism comprising a 16S rDNA sequence containing a sequence with more than 87% homology to SEQ. ID No: 1.

2. The microorganism according to claim 1, wherein said 16S rDNA sequence fragment comprises SEQ ID No: 1.

3. The microorganism according to claim 1, deposited at ATCC under accession no. PTA-9150.

4. A derivative or mutant of the microorganism of claim 1 comprising a 16S rDNA sequence containing a sequence with more than 87% homology to SEQ ID No: 1.

5. The microorganism of claim 4 wherein said 16S rDNA sequence comprises SEQ ID No: 1.

6. A method of producing molecular hydrogen (H₂), said method comprising culturing the microorganism of claim 1 under conditions allowing hydrogen production.

7. The method of claim 6 wherein said conditions comprise the presence of a metallosilicate, such as zeolite.

8. The method of claim 6 wherein said conditions comprise an aqueous environment containing gram amounts of added alkali phosphates, yeast extract, malt extract, and/or a protein hydrolysate extract, e.g. tryptone or peptone; or
wherein said conditions comprise an aqueous environment containing milli- or microgram amounts of added inorganic salts, such as calcium, magnesium, manganese, iron, selenium, molybdenum, nickel and/or zinc, or any combination thereof; or
wherein said conditions comprise an aqueous environment containing defined amounts of redox-active compounds and/or compounds with either antioxidant or oxidant chemical characteristics, such as ascorbic acid, N-acetyl cysteine, methionine, cysteine, glutathione, and/or hydrogen peroxide.

9. The method of claim 6 wherein said conditions comprise a gas phase above an aqueous environment that is continuously flushed with defined amounts of a gas, such as the noble gas argon.

10. The method of claim 6 wherein a gas phase above the aqueous environment is flushed at defined time points with defined amounts of a gas, preferentially the noble gas argon.

11. The method of claim 6 wherein said conditions comprise an aqueous environment that is continuously bubbled with defined amounts of a gas, such as the noble gas argon.

12. The method of claim 6 wherein said conditions comprise an aqueous environment that is flushed at defined time points with defined amounts of a gas, such as the noble gas argon.

13. The method of claim 6 wherein said conditions comprise an environment maintained at a temperature below 45° C.; or
wherein said conditions comprise an environment that is maintained at a constant pH of between 4.5 and 7.5.

14. The method of claim 6 wherein said conditions comprise a continuously supplied liquid feedstock derived from the group consisting of monosaccharides, disaccharides, polysaccharides, alcoholic sugars, polyhydroxyalcohols, amino acids, fatty acids, and combinations thereof.

15. The method of claim 14 wherein the mono- and disaccharides are glucose, sucrose, maltose, cellobiose and/or other saccharides containing glucose units or any combination thereof; or
wherein the feedstock contains arabinose, xylose, galactose, rhamnose, sorbitol and/or mannitol or combinations thereof; or
wherein the feedstock contains polyhydroxyalcohols, e.g. glycerol, monoacylglycerol and/or diacylglycerol or any combination thereof.

16. The method of claim 6 wherein the conditions comprise generation of carbon dioxide which is chemically bound with the help of an alkali metal liquid matrix, such as sodium hydroxide (NaOH), and/or a solid matrix, such as soda lime.

17. A method of genetically engineering the microorganism of claim 1, said method comprising transformation of said microorganism with the use of one or more DNA-, RNA- or PNA-based vehicles, such as plasmids, bacteriophages or viruses, optionally further comprising screening said transformed microorganism for increased hydrogen production rates and/or output.

18. A method of mutagenizing the microorganism of claim 1, said method comprising the treatment of said microorganism with a mutagen, optionally further comprising screening said treated microorganism for increased hydrogen production rates and/or output.

19. The method of claim 18 wherein the mutagen is UV or ionizing irradiation, a deaminating agent, an alkylating agent, sodium azide, an intercalating agent, or phage or transposon mediated mutagenesis.

20. The method of claim 19 wherein the deaminating agent is nitrous acid, the alkylating agent is methyl-N-nitrosoguanidine (MNNG) and the intercalating agent is ethidium bromide.


SEQUENCE LISTING (3 sequences)

SEQ ID NO: 1 - DNA, 671 bp, Enterobacter sp.
cacatcgcatacgtcgcagaccaaagtgggggaccttcgggcctcatgccatcagatgtgcccagatgggattagctagtaggtggggtaatggctcacctaggcgacgatccctagctggtctgagaggatgaccagccacactggaactgatacacggtccagactcctacgggaggcagcagtggggaatattgatttatgggcgcaagcctgatgcagccatgccgcgtgtatgaacaaggccttccgattgtaaattgctttctccgaataggaaggcctgctggttaataaccttgcggattgactttactcgcaaacgaagcaccggctaactccgtgccttaagcccttcctcctcggagggtgcactttttaatccgaattactggttcttaagcgcacgctggctgcctgtcgcttgcgatgtgaaatccccgggctccacctgggaactgcattcgaaactggaccgctagagtcttgtagaggggggtggaattcctcgtgtaccggtgaaatgcgtacagatctggaagaataccccccaccaaggcggccccctggacaaagactgactctcaggtgcaaaaccgtggggagcccacttgattatataccctggtagtccactccgctaccgatgtcaacttgattcccccctccaa

SEQ ID NO: 2 - DNA, 20 bp, Artificial Sequence
  PCR Primer
tggagggggataactactgg

SEQ ID NO: 3 - DNA, 18 bp, Artificial Sequence
  PCR Primer
ggcacaacctccaagtcg